(19) United States
(12) Patent Application Publication
(10) Pub. No.: US 2010/0197797 A1
(43) Pub. Date: Aug. 5, 2010

(54) METHODS OF MANAGING FIBROMYALGIA USING MILNACIPRAN

(76) Inventors: Srinivas G. Rao, Encinitas, CA (US); Michael R. Gendreau, Poway, CA (US); Mahendra G. Dedhiya, Pomona, NY (US)

Correspondence Address: Forest Laboratories, Inc.;Attn: Charles S. Ryan, 500 COMMACK ROAD, Commack, NY, 11725, US

(21) Appl. No.: 12/700,994

(22) Filed: Feb. 5, 2010

Related U.S. Application Data
(60) Provisional application No. 61/150,140, filed on Feb. 5, 2009.

(51) Int. Cl. A61K 31/165; A61P 29/00

(52) U.S. Cl. 514/620

(57) ABSTRACT

The present invention relates to compositions comprising milnacipran or a pharmaceutically acceptable salt thereof (e.g., milnacipran hydrochloride) and methods for managing fibromyalgia comprising administering milnacipran or a pharmaceutically acceptable salt thereof (e.g., milnacipran hydrochloride). The present invention also relates to titration packs comprising dosage forms (e.g., tablets) comprising milnacipran or a pharmaceutically acceptable salt thereof (e.g., milnacipran hydrochloride) for oral administration. The titration packs enable patient compliance with a regime of changing dosage of the milnacipran or pharmaceutically acceptable salt thereof (e.g., milnacipran hydrochloride).

### CROSS REFERENCE TO RELATED APPLICATIONS

This application claims priority under 35 U.S.C. §119, based on U.S. Provisional Application Ser. No. 61/150,140 filed on Feb. 5, 2009, which is hereby incorporated by reference in its entirety.

### FIELD OF THE INVENTION

The present invention relates to compositions comprising milnacipran or a pharmaceutically acceptable salt thereof (e.g., milnacipran hydrochloride) and methods for managing fibromyalgia comprising administering milnacipran or a pharmaceutically acceptable salt thereof (e.g., milnacipran hydrochloride). The present invention also relates to titration packs comprising dosage forms (e.g., tablets) comprising milnacipran or a pharmaceutically acceptable salt thereof (e.g., milnacipran hydrochloride) for oral administration. The titration packs enable patient compliance with a regime of changing dosage of the milnacipran or pharmaceutically acceptable salt thereof (e.g., milnacipran hydrochloride).

### BACKGROUND OF THE INVENTION

Milnacipran is a norepinephrine-serotonin reuptake inhibitor (NSRI), which inhibits the uptake of both norepinephrine (NE) and serotonin (5-HT), with an NE to 5-HT ratio of 2:1 (Moret et al., Neuropharmacology, 24:1211-1219, 1985; Palmier et al., Eur. J. Clin. Pharmacol., 37:235-238, 1989) but does not affect the uptake of dopamine. Milnacipran and methods of treatment using milnacipran are disclosed, for example, in U.S. Pat. Nos. 4,478,836, 6,602,911, 6,635,675 and 6,992,110.

Adverse events associated with the administration of immediate release formulations of milnacipran may include, for example, nausea, vomiting, headache, tremulousness, anxiety, panic attack, palpitations, urinary retention, orthostatic hypotension, diaphoresis, chest pain, rash, weight increase, back pain, constipation, diarrhea, vertigo, increased sweating, agitation, hot flushes, fatigue, somnolence, dyspepsia, dysuria, dry mouth, abdominal pain, and insomnia. Due to the incidence of adverse events, patients often do not tolerate high-doses of milnacipran and patient compliance may often be affected.

Thus, there is an existing and continual need for methods of administering milnacipran, or pharmaceutically acceptable salts thereof, (e.g., milnacipran hydrochloride) which facilitate a better patient compliance.

### SUMMARY OF THE INVENTION

According to some embodiments, the present invention provides methods of managing fibromyalgia in a patient in need thereof comprising administering a dose of about 10 mg to about 50 mg of milnacipran or a pharmaceutically acceptable salt thereof per day in a patient with a creatinine clearance of about 5 to about 29 ml/min.

According to some embodiments, the present invention provides methods of managing fibromyalgia in a patient in need thereof comprising providing about 10 mg to about 50 mg of milnacipran or a pharmaceutically acceptable salt thereof and informing the patient or a health care worker that about 50 mg/day of milnacipran or a pharmaceutically acceptable salt thereof should be administered in a patient with a creatinine clearance of about 5 to about 29 ml/min.

### BRIEF DESCRIPTION OF THE DRAWINGS

FIG. 1 shows patients in Study 1 achieving various levels of pain relief with concurrent ratings of being much or very much improved on the Patient Global Impression of Change (PGIC). (MLN=milnacipran).

FIG. 2 shows patients in Study 2 achieving various levels of pain relief with concurrent ratings of being much or very much improved on the Patient Global Impression of Change (PGIC). (MLN=milnacipran).

FIG. 3 represents a titration pack and a dosage titration schedule comprising milnacipran hydrochloride tablets of three different strengths; 12.5 mg, 25 mg, and 50 mg.

FIG. 4 represents a titration pack and a dosage titration schedule comprising milnacipran hydrochloride tablets of three different strengths; 12.5 mg, 25 mg, and 50 mg.

FIG. 5 represents a titration pack and a dosage titration schedule comprising milnacipran hydrochloride tablets of three different strengths; 12.5 mg, 25 mg, and 50 mg.

FIG. 6 represents a titration pack and a dosage titration schedule comprising milnacipran hydrochloride tablets of four different strengths; 12.5 mg, 25 mg, 50 mg and 100 mg.

### DETAILED DESCRIPTION OF THE INVENTION

Milnacipran hydrochloride is a selective norepinephrine and serotonin reuptake inhibitor; it inhibits norepinephrine uptake with greater potency than serotonin. It is a racemic mixture with the chemical name: (±)-[1R(S),2S(R)]-2-(aminomethyl)-N,N-diethyl-1-phenylcyclopropanecarboxamide hydrochloride. The structural formula of milnacipran hydrochloride is:

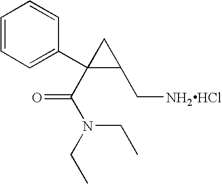

Milnacipran hydrochloride is a white to off-white crystalline powder with a melting point of 179° C. It is freely soluble in water, methanol, ethanol, chloroform, and methylene chloride and sparingly soluble in diethyl ether. It has an empirical formula of C₁₅H₂₃ClN₂O and a molecular weight of 282.8 g/mol.

The present invention relates to compositions comprising milnacipran or a pharmaceutically acceptable salt thereof (e.g., milnacipran hydrochloride) and methods of managing fibromyalgia by administering milnacipran or a pharmaceutically acceptable salt thereof (e.g., milnacipran hydrochloride). The present invention also relates to titration packs comprising dosage forms (e.g., tablets) comprising milnacipran or a pharmaceutically acceptable salt thereof (e.g., milnacipran hydrochloride) for oral administration.

Compositions

In one aspect, the present invention provides compositions comprising milnacipran or a pharmaceutically acceptable salt thereof (e.g., milnacipran hydrochloride) for management of fibromyalgia.

In some embodiments, the compositions comprise milnacipran hydrochloride. The compositions may comprise about 10 mg to about 200 mg of milnacipran hydrochloride. For example, the compositions may comprise about 10 mg, about 12.5 mg, about 20 mg, about 25 mg, about 50 mg, about 75 mg, about 100 mg, about 125 mg, about 150 mg, about 175 mg or about 200 mg of milnacipran hydrochloride.

In other embodiments, the compositions may consist essentially of milnacipran or a pharmaceutically acceptable salt thereof (e.g., milnacipran hydrochloride). In such embodiments, milnacipran or a pharmaceutically acceptable salt thereof (e.g., milnacipran hydrochloride) is the only active ingredient or therapeutic agent. The compositions may further comprise inactive ingredients such as one or more pharmaceutically acceptable carriers, excipients or diluents. For example, the compositions may consist essentially of about 10 mg, about 12.5 mg, about 20 mg, about 25 mg, about 50 mg, about 75 mg, about 100 mg, about 125 mg, about 150 mg, about 175 mg or about 200 mg of milnacipran or a pharmaceutically acceptable salt thereof (e.g., milnacipran hydrochloride). In exemplary embodiments, the compositions consist essentially of milnacipran hydrochloride.

In some embodiments, the present invention provides compositions for management of fibromyalgia that comprise from about 10 mg to about 200 mg of milnacipran or a pharmaceutically acceptable salt thereof (e.g., milnacipran hydrochloride) and provide a mean AUC for milnacipran in patients with a creatinine clearance of from about 50 to about 80 ml/min of about 1.1 to about 1.2 times greater than mean AUC for milnacipran in patients with a renal clearance of greater than about 80 ml/min. For example, the compositions may comprise about 10 to about 200 mg (e.g., about 12.5 mg, about 25 mg, about 50 mg or about 100 mg) of milnacipran or a pharmaceutically acceptable salt thereof (e.g., milnacipran hydrochloride) and provide a mean AUC for milnacipran in patients with a creatinine clearance of from about 50 to about 80 ml/min, which is about 10% to about 20% greater than mean AUC for milnacipran in patients with a creatinine clearance of greater than about 80 ml/min. In some examples, the mean AUC may be increased by about 10%, about 11%, about 12%, about 13%, about 14%, about 15%, about 16%, about 17%, about 18%, about 19% or about 20%.

In some embodiments, the present invention provides compositions for management of fibromyalgia that comprise from about 10 mg to about 200 mg of milnacipran or a pharmaceutically acceptable salt thereof (e.g., milnacipran hydrochloride) and provide a mean AUC for milnacipran in patients with a creatinine clearance of from about 30 to about 49 ml/min of about 1.4 to about 1.7 times greater than mean AUC for milnacipran in patients with a renal clearance of greater than about 80 ml/min. For example, the compositions may comprise about 10 to about 200 mg (e.g., about 12.5 mg, about 25 mg, about 50 mg or about 100 mg) of milnacipran or a pharmaceutically acceptable salt thereof (e.g., milnacipran hydrochloride) and provide a mean AUC for milnacipran in patients with a creatinine clearance of from about 30 to about 49 ml/min, which is about 40% to about 70% greater than mean AUC for milnacipran in patients with a creatinine clearance of greater than about 80 ml/min. In some examples, the mean AUC may be increased by about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60% or about 65%.

In some embodiments, the present invention provides compositions for management of fibromyalgia that comprise from about 10 mg to about 200 mg of milnacipran or a pharmaceutically acceptable salt thereof (e.g., milnacipran hydrochloride) and provide a mean AUC for milnacipran in patients with a creatinine clearance of from about 5 to about 29 ml/min of about 1.8 to about 3.5 times greater than mean AUC for milnacipran in patients with a renal clearance of greater than about 80 ml/min. For example, the compositions may comprise about 10 to about 200 mg (e.g., about 12.5 mg, about 25 mg, about 50 mg or about 100 mg) of milnacipran or a pharmaceutically acceptable salt thereof (e.g., milnacipran hydrochloride) and provide a mean AUC for milnacipran in patients with a creatinine clearance of from about 5 to about 29 ml/min, which is about 100% to about 250% greater than mean AUC for milnacipran in patients with a creatinine clearance of greater than about 80 ml/min. In some examples, the mean AUC may be increased by about 150%, about 160%, about 170%, about 180%, about 190%, about 200% or about 220%.

The pharmaceutically acceptable salts of milnacipran include salts with inorganic or organic acids, such as hydrochloric acid, hydrobromic acid, phosphoric acid, nitric acid, sulfuric acid, methanesulfonic acid, p-toluenesulfonic acid, acetic acid, fumaric acid, succinic acid, lactic acid, mandelic acid, malic acid, citric acid, tartaric acid or maleic acid. In addition, compounds containing a carboxy group or other acidic group may be used. In some examples, the compounds may be converted into a pharmaceutically acceptable addition salt with inorganic or organic bases including, but not limited to, sodium hydroxide, potassium hydroxide, ammonia, cyclohexylamine, dicyclohexyl-amine, ethanolamine, diethanolamine and triethanolamine.

The compositions described above may be administered through different routes, such as, oral, inhalation, transdermal, rectal, transmucosal, intestinal or parenteral administration (e.g, intramuscular, subcutaneous or intravenous injections). In some embodiments, the compositions may comprise milnacipran or a pharmaceutically acceptable salt thereof in an admixture or mixture with one or more pharmaceutically acceptable carriers, excipients or diluents.

Milnacipran or a pharmaceutically acceptable salt thereof (e.g., milnacipran hydrochloride) can be formulated for oral administration using pharmaceutically acceptable carriers well known in the art. Such carriers may be used to formulate the compositions described above as tablets, pills, dragees, capsules, liquids, gels, syrups, slurries, suspensions and the like, for oral administration. Suitable excipients include, but are not limited to, fillers such as sugars, including lactose, sucrose, mannitol, or sorbitol; cellulose preparations, such as maize starch, wheat starch, rice starch, potato starch, gelatin, gum tragacanth, methylcellulose, hydroxypropylmethylcellulose, sodium carboxymethylcellulose, and/or polyvinylpyrrolidone (PVP). Disintegrants such as cross-linked polyvinylpyrrolidone, agar, or alginic acid or a salt thereof (e.g., sodium alginate) may be added.

In some embodiments, milnacipran or a pharmaceutically acceptable salt thereof (e.g., milnacipran hydrochloride) may be formulated as push-fit capsules made of gelatin, as well as soft, sealed capsules made of gelatin and a plasticizer, such as glycerol or sorbitol. The push-fit capsules may contain milnacipran or a pharmaceutically acceptable salt thereof (e.g., milnacipran hydrochloride) in an admixture with a filler such as lactose, a binder such as starches, and/or a lubricant such as talc or magnesium stearate and, optionally, stabilizers. In soft capsules, the milnacipran or a pharmaceutically acceptable salt thereof (e.g., milnacipran hydrochloride) may be dissolved or suspended in suitable liquids, such as fatty oils, liquid paraffin, or liquid polyethylene glycols. In addition, stabilizers may be added.

In exemplary embodiments, milnacipran or a pharmaceutically acceptable salt thereof (e.g., milnacipran hydrochloride) is formulated for oral administration as film-coated tablets. The tablets may comprise about 12.5 mg, about 25 mg, about 50 mg, or about 100 mg milnacipran or a pharmaceutically acceptable salt thereof (e.g., milnacipran hydrochloride). In further embodiments, the tablets may also contain dibasic calcium phosphate, povidone, carboxymethylcellulose calcium, colloidal silicon dioxide, magnesium stearate, or talc or combinations thereof as inactive ingredients.

In further embodiments, the following inactive ingredients may be present as components of a film coat:

12.5 mg: FD&C Blue #2 Aluminum Lake, hypromellose, polyethylene glycol, titanium dioxide or combinations thereof;

25 mg: Hypromellose, polyethylene glycol, titanium dioxide or combinations thereof;

50 mg: Hypromellose, polyethylene glycol, titanium dioxide or combinations thereof;

100 mg: FD&C Red #40 Aluminum Lake, hypromellose, polyethylene glycol, titanium dioxide or combinations thereof.

Milnacipran or a pharmaceutically acceptable salt thereof (e.g., milnacipran hydrochloride) may be administered as injections using aqueous solutions, preferably, physiologically compatible buffers (e.g., Hanks's solution, Ringer's solution, or physiological saline buffer). For transmucosal administration, penetrants appropriate to the barrier to be permeated may be used in the compositions. For buccal administration, the compositions may take the form of tablets or lozenges formulated in conventional manner. For administration by inhalation, the milnacipran or pharmaceutically acceptable salt thereof (e.g., milnacipran hydrochloride) may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of gelatin, for example, for use in an inhaler or insufflator may be formulated containing a powder mix of the milnacipran or a pharmaceutically acceptable salt thereof and a suitable powder base such as lactose or starch.

Milnacipran or a pharmaceutically acceptable salt thereof may be provided for parenteral administration as a powder for constitution with a suitable vehicle, e.g., sterile pyrogen-free water, before use. In some examples, the compositions may be provided in unit dosage form, e.g., in ampoules or in multi-dose containers, with an added preservative for parenteral administration. The compositions may be formulated as suspensions, solutions or emulsions in oily or aqueous vehicles, and may contain formulatory agents such as suspending, stabilizing and/or dispersing agents. Milnacipran or a pharmaceutically acceptable salt thereof (e.g., milnacipran hydrochloride) may also be formulated in rectal compositions such as suppositories or retention enemas, e.g., containing conventional suppository bases such as cocoa butter or other glycerides.

In another aspect, the present invention provides titration packs comprising dosage forms comprising milnacipran or a pharmaceutically acceptable salt thereof (e.g., milnacipran hydrochloride) for oral administration. The titration packs enable dosage titration of milnacipran or a pharmaceutically acceptable salt thereof (e.g., milnacipran hydrochloride) over a period of time. The titration packs may thus facilitate better patient compliance and reduction of medication error through efficient administration of milnacipran tablets. In some embodiments, the titration packs comprise compositions or dosage forms comprising milnacipran or a pharmaceutically acceptable salt thereof (e.g., milnacipran hydrochloride) as described above.

In some embodiments, the titration packs comprise dosage forms comprising milnacipran or a pharmaceutically acceptable salt thereof (e.g., milnacipran hydrochloride) for oral administration.

In exemplary embodiments, the titration packs comprise milnacipran hydrochloride. In additional exemplary embodiments, the titration packs comprise (1S,2R)-2-aminomethyl-1-phenyl-N,N-diethylcyclopropanecarboxamide hydrochloride. In further embodiments, the titration packs comprise substantially pure (1S,2R)-2-aminomethyl-1-phenyl-N,N-diethylcyclopropanecarboxamide hydrochloride. In additional embodiments, the titration packs comprise a mixture (e.g., a non-racemic mixture) of (1S,2R)-2-aminomethyl-1-phenyl-N,N-diethylcyclopropanecarboxamide hydrochloride and (1R,2S)-2-aminomethyl-1-phenyl-N,N-diethylcyclopropanecarboxamide hydrochloride.

In some embodiments, the present invention provides a titration pack for enabling compliance with a regimen of changing dosage of milnacipran hydrochloride over a period of time, the pack comprising: a backing having an array of receivers, said array including a plurality of columns and a plurality of rows; a plurality of sets of milnacipran hydrochloride tablets, each tablet in a set having a common dose of milnacipran hydrochloride and a different dose than a tablet of a different set, each set being disposed in receivers of one of an adjacent row and an adjacent column; different sets of milnacipran hydrochloride tablets are disposed in different rows, each row being indicated as a successive day or week, each column being indicated as a different day of the day or week, sets of milnacipran hydrochloride tablets having increased doses are disposed in receivers of rows indicated as successive days or weeks; and indicia disposed adjacent the columns and rows for displaying common days and successive weeks.

For example, the titration packs comprise tablets of milnacipran hydrochloride. According to some embodiments, the titration packs comprise milnacipran hydrochloride tablets of varying strengths. The tablets may be arranged in rows or columns in order of increasing dosage. In some embodiments, the titration packs comprise milnacipran hydrochloride tablets of at least three different strengths: about 12.5 mg; about 25 mg and about 50 mg. In other embodiments, the titration packs comprise milnacipran tablets of at least four different strengths: about 12.5 mg; about 25 mg; about 50 mg; and about 100 mg. In yet other embodiments, the titration packs, comprise milnacipran tablets of at least five different strengths: about 12.5 mg; about 25 mg; about 50 mg; about 100 mg; and about 200 mg.

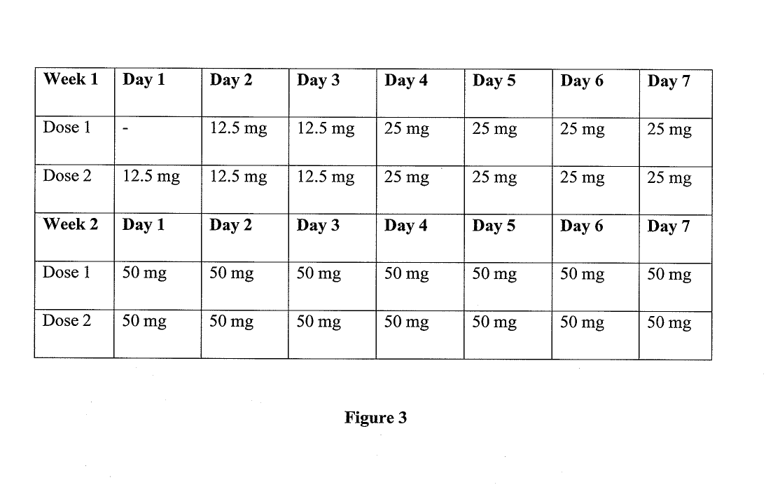

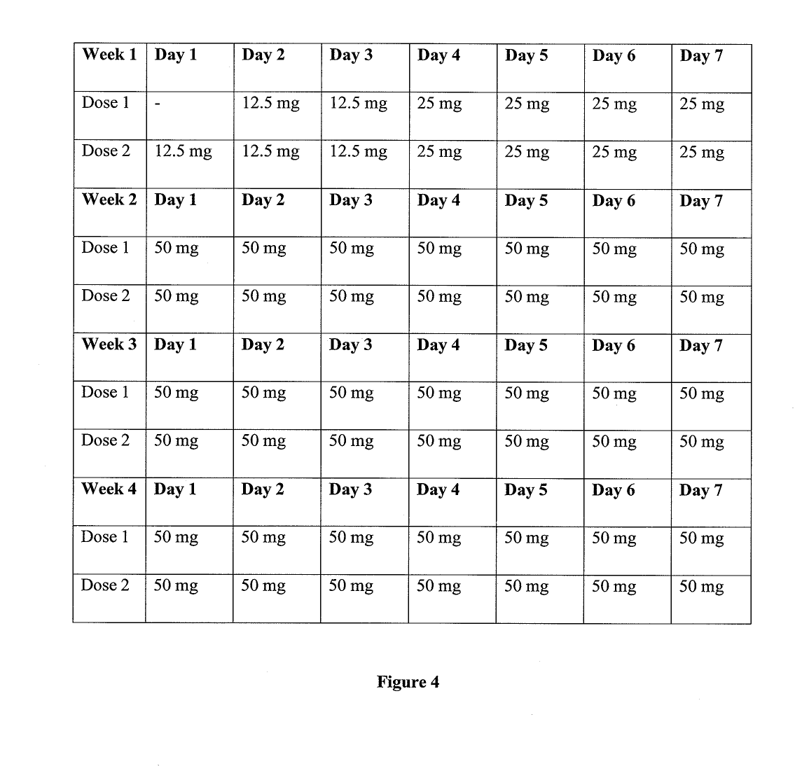

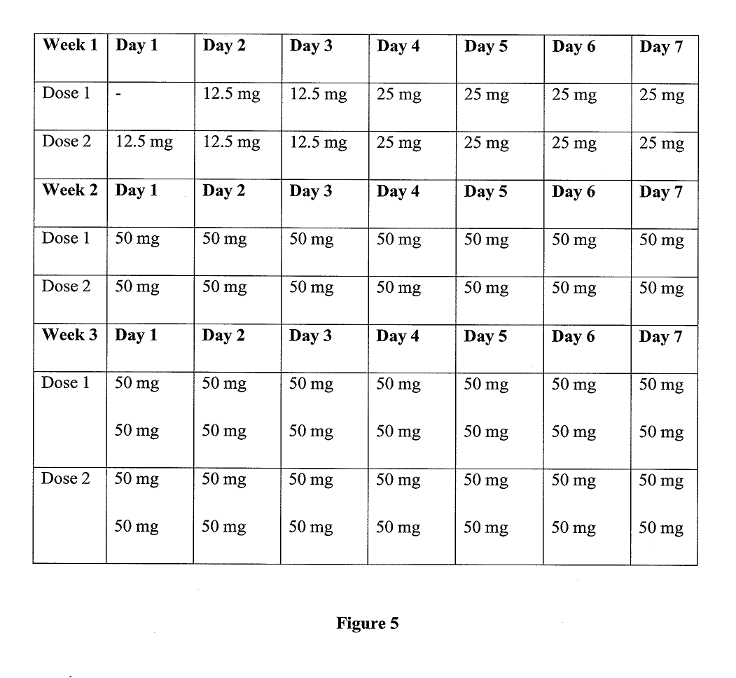

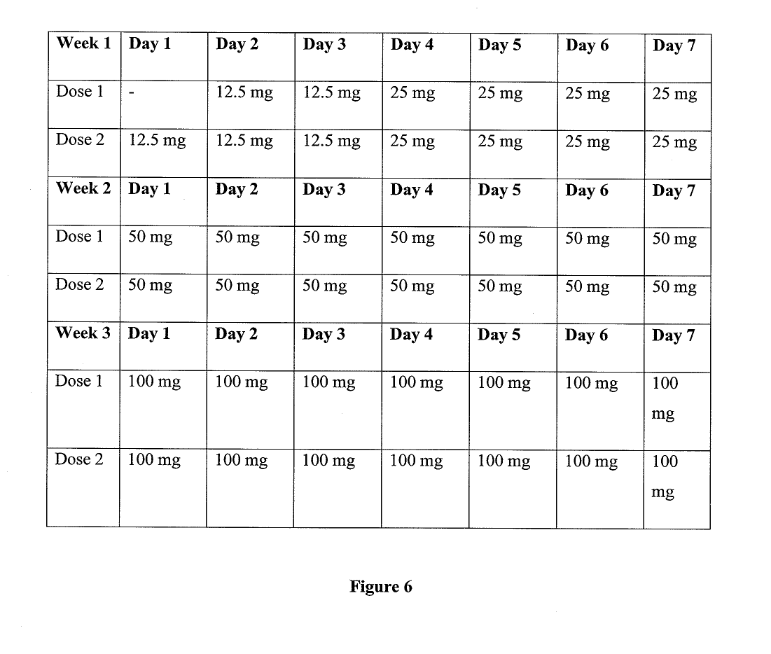

In exemplary embodiments, the titration packs comprise milnacipran hydrochloride tablets and a dosage titration schedule as shown in FIG. 3. In other exemplary embodiments, the titration packs comprise milnacipran hydrochloride tablets and a dosage titration schedule as shown in FIG. 4. In still other exemplary embodiments, the titration pack comprises milnacipran hydrochloride tablets and a dosage titration schedule as shown in FIG. 5. In other examples, the titration packs comprise milnacipran hydrochloride tablets and a dosage titration schedule as shown in FIG. 6.

Methods

In another aspect, the present invention provides methods of managing fibromyalgia comprising administering milnacipran or a pharmaceutically acceptable salt thereof (e.g., milnacipran hydrochloride). The methods include administering compositions or dosage forms comprising milnacipran or a pharmaceutically acceptable salt thereof (e.g., milnacipran hydrochloride) as described above. In some embodiments, the methods include providing titration packs comprising milnacipran or a pharmaceutically acceptable salt thereof (e.g., milnacipran hydrochloride) as described above.

In exemplary embodiments, the methods comprise administering milnacipran hydrochloride. The methods include administering milnacipran alone or in combination with other therapeutic agents. In exemplary embodiments, the methods comprise administering a composition consisting essentially of milnacipran hydrochloride. Milnacipran hydrochloride is the only active ingredient or therapeutic agent in such compositions.

In some embodiments, the methods comprise administering milnacipran or a pharmaceutically acceptable salt thereof (e.g., milnacipran hydrochloride) in a dose of about 10 mg to about 200 mg per day. In exemplary embodiments, the daily dose may be about 10 mg, about 12.5 mg, about 20 mg, about 25 mg, about 50 mg, about 75 mg, about 100 mg, about 125 mg, about 150 mg, about 175 mg or about 200 mg. The daily dose may be administered in two to five divided doses. In specific embodiments, the daily dose may be administered in two divided doses per day. For example, about 50 mg/day may be administered in two divided doses of about 25 mg. In other examples, about 100 mg/day may be administered in two divided doses of about 50 mg.

In some embodiments, the methods comprise administering milnacipran or a pharmaceutically acceptable salt thereof (e.g., milnacipran hydrochloride) at a starting dose of about 12.5 mg, and then increasing the dosage to about 25 mg/day, then to about 50 mg/day, then up to a target dose of up to about 100 mg/day or about 200 mg/day in subsequent weeks.

In certain embodiments, the methods comprise administering milnacipran or a pharmaceutically acceptable salt thereof (e.g., milnacipran hydrochloride) at a starting dose of about 12.5 mg in the first week, and then increasing the dosage to about 25 mg/day, then to about 50 mg/day, then up to a target dose of up to about 100 mg/day or about 200 mg/day in subsequent weeks. The titration packs comprise dosage forms (e.g., tablets) comprising milnacipran or a pharmaceutically acceptable salt thereof (e.g., milnacipran hydrochloride) of varying strengths to enable such dosage titration. For example, the titration packs may comprise milnacipran hydrochloride tablets. The tablets may be arranged in any arrangement, such as in rows or columns in order of increasing dosage, so as to ensure or increase ease of administration and patient compliance.

In some embodiments, the methods consist essentially of administering milnacipran or a pharmaceutically acceptable salt thereof (e.g., milnacipran hydrochloride) in a dose of about 10 mg to about 200 mg per day. In such methods, milnacipran or a pharmaceutically acceptable salt thereof (e.g., milnacipran hydrochloride) is the only active ingredient or therapeutic agent being administered. In exemplary embodiments, the daily dose may be about 10 mg, about 12.5 mg, about 20 mg, about 25 mg, about 50 mg, about 75 mg, about 100 mg, about 125 mg, about 150 mg, about 175 mg or about 200 mg. The daily dose may be administered in two to five divided doses. In specific embodiments, the daily dose may be administered in two divided doses per day. For example, about 50 mg/day may be administered in two divided doses of about 25 mg. In other examples, about 100 mg/day may be administered in two divided doses of about 50 mg.

In certain embodiments, milnacipran or a pharmaceutically acceptable salt thereof (e.g., milnacipran hydrochloride) is indicated for the management of fibromyalgia.

In certain embodiments, milnacipran or a pharmaceutically acceptable salt thereof (e.g., milnacipran hydrochloride) is not approved for use in pediatric patients.

In some embodiments, milnacipran or a pharmaceutically acceptable salt thereof (e.g., milnacipran hydrochloride) is given orally without food.

In other embodiments, milnacipran or a pharmaceutically acceptable salt thereof (e.g., milnacipran hydrochloride) is given orally with food. In some embodiments, taking milnacipran hydrochloride with food may improve the tolerability of the drug.

In certain embodiments, the recommended dose of milnacipran or a pharmaceutically acceptable salt thereof (e.g., milnacipran hydrochloride) is 100 mg/day (e.g., 50 mg twice daily).

In certain embodiments, dosing should be titrated according to the following schedule:

Day 1: 12.5 mg once
Days 2-3: 25 mg/day (e.g, 12.5 mg twice daily)
Days 4-7: 50 mg/day (e.g., 25 mg twice daily)
After Day 7: 100 mg/day (e.g., 50 mg twice daily)

In certain embodiments, based on individual patient response, the dose may be increased to 200 mg/day (e.g., 100 mg twice daily).

In certain embodiments, administration of milnacipran or a pharmaceutically acceptable salt thereof (e.g., milnacipran hydrochloride) should be tapered and not abruptly discontinued after extended use.

In a further aspect, the present invention provides methods of treatment and titration packs comprising milnacipran or a pharmaceutically acceptable salt thereof (e.g., milnacipran hydrochloride) for oral administration to aid the patients in titrating milnacipran dosage over time. The titration packs and methods disclosed herein optimize the administration and dosage titration of milnacipran or a pharmaceutically acceptable salt thereof (e.g., milnacipran hydrochloride) and thus provide a better patient compliance with a regime of changing dosage of milnacipran.

In this regard, non-adherence to fibromyalgia medication therapy and medication error are considerable problems. These problems can be significantly reduced by providing fibromyalgia patients with a titration pack comprising dosages forms of milnacipran or a pharmaceutically acceptable salt thereof (e.g., milnacipran hydrochloride) of different strength. Providing these compounds in this fashion makes therapy simple because it increases convenience and eliminates confusion in preparing appropriate dosages. These advantages are especially significant where treatments often come in multiple dosage units and must be diluted to specific concentrations suitable for treating patients. As discussed previously, this poses several problems.

According to some embodiments, the methods of treatment comprise administering milnacipran or a pharmaceutically acceptable salt thereof (e.g., milnacipran hydrochloride) starting at a low dose of about 12.5 mg/day in the first week and titrating the dosage up to a target dose of up to about 100 mg/day in subsequent days or weeks. In other embodiments, depending on the patient response, dosage of milnacipran or a pharmaceutically acceptable salt thereof (e.g., milnacipran hydrochloride) is titrated to about 200 mg/day or even a higher tolerated dose of up to about 400 mg in subsequent days or weeks.

In other embodiments, the methods may comprise administering milnacipran or a pharmaceutically acceptable salt thereof (e.g., milnacipran hydrochloride) in a dose of about 10 mg to about 50 mg per day in patients with a creatinine clearance of about 5 to about 29 ml/min. For example, the methods may comprise administering a dose of about 12.5 mg on Day 1, about 25 mg on Days 2 and 3, about 50 mg on Day 4 and maintaining the dose at about 50 mg per day after Day 4. The dose of about 50 mg/day may be administered in two divided doses of about 25 mg. For example, the methods may comprise administering a dose of about 10 to about 50 mg of milnacipran hydrochloride to patients with a creatinine clearance of about 5 to about 29 ml/min.

In some embodiments, the present invention provides methods of managing fibromyalgia in a patient in need thereof comprising providing instructions on dosage and administration of milnacipran or a pharmaceutically acceptable salt thereof (e.g., milnacipran hydrochloride). In exemplary embodiments, the methods may comprise informing the patient or a health care worker that a recommended dosage of milnacipran ranges from about 100 mg to about 200 mg/day and advising the patient or health care worker to titrate the dosage according to the following schedule: Day 1: about 12.5 mg; Days 2-3: about 25 mg/day, Days 4-7: about 50 mg/day and after Day 7: about 100 mg/day.

In some embodiments, the methods comprise providing instructions on dose adjustment in patients with a creatinine clearance of about 5 to about 29 ml/min. The methods may comprise informing the patient or health care worker a recommended dose in such patients is about 50 mg/day. For example, the methods may comprise informing the patient or a health care worker that a recommended dose in a patient with a creatinine clearance of about 5 to about 29 ml/min is about 25 mg twice daily.

In specific embodiments, the methods comprise providing instructions on dosage and administration of milnacipran or a pharmaceutically acceptable salt thereof (e.g., milnacipran hydrochloride); informing the patient or a health care worker that a recommended dosage of milnacipran ranges from about 100 mg to about 200 mg/day and advising the patient or health care worker to titrate the dosage according to the following schedule: Day 1: about 12.5 mg; Days 2-3: about 25 mg/day, Days 4-7: about 50 mg/day and Days 8-14: about 100 mg/day.

In still other embodiments, the methods comprise providing instructions on dosage and administration of milnacipran or a pharmaceutically acceptable salt thereof (e.g., milnacipran hydrochloride) to the patient or a health care worker; informing the patient or health care worker that a recommended dosage of the milnacipran or pharmaceutically acceptable salt thereof (e.g., milnacipran hydrochloride) ranges from about 100 mg to about 200 mg/day and advising the patient or health care worker to titrate the dosage according to the following schedule: Day 1: about 12.5 mg; Days 2-3: about 25 mg/day, Days 4-7: about 50 mg/day and Days 8-28: about 100 mg/day.

In some embodiments, the patient or a health care worker is advised to titrate the dosage according to the following schedule: Day 1: about 12.5 mg; Days 2-3: about 25 mg/day, Days 4-7: about 50 mg/day and after Day 7: about 100 mg/day; and further, depending on the individual response, the patient or health care worker is advised to increase dosage to about 200 mg/day.

In exemplary embodiments, the patient or health care worker is advised to titrate the dosage of milnacipran hydrochloride according to the following schedule: Day 1: about 12.5 mg requiring a single administration of 12.5 mg milnacipran hydrochloride tablet; Days 2-3: about 25 mg/day requiring administration of a 12.5 mg milnacipran hydrochloride tablet twice a day, Days 4-7: about 50 mg/day requiring administration of a 25 mg milnacipran hydrochloride tablet twice a day, and after Day 7: about 100 mg/day requiring administration of a 50 mg milnacipran hydrochloride tablet twice a day.

In further embodiments, the patient or health care worker is advised to titrate the dosage of milnacipran hydrochloride according to the following schedule: Day 1: about 12.5 mg requiring a single administration of a 12.5 mg tablet; Days 2-3: about 25 mg/day requiring administration of a 12.5 mg milnacipran hydrochloride tablet twice a day, Days 4-7: about 50 mg/day requiring administration of a 25 mg milnacipran hydrochloride tablet twice a day, and after Day 7: about 100 mg/day requiring administration of a 50 mg milnacipran hydrochloride tablet twice a day, and if required, optionally increasing the dosage to about 200 mg/day requiring the administration of 100 mg milnacipran hydrochloride twice a day (e.g., administered as two 50 mg milnacipran hydrochloride tablets twice a day).

In yet other embodiments, the patient or health care worker is advised to titrate the dosage of milnacipran hydrochloride according to the following schedule: Day 1: about 12.5 mg requiring a single administration of a 12.5 mg milnacipran hydrochloride tablet; Days 2-3: about 25 mg/day requiring administration of a 12.5 mg milnacipran hydrochloride tablet twice a day, Days 4-7: about 50 mg/day requiring administration of a 25 mg milnacipran hydrochloride tablet twice a day, and after Day 7: about 100 mg/day requiring administration of a 50 mg milnacipran hydrochloride tablet twice a day, and if required, optionally increasing the dosage to about 200 mg/day requiring the administration of a 100 mg milnacipran hydrochloride tablet twice a day.

In some embodiments, the present invention provides methods of managing fibromyalgia in a patient diagnosed with fibromyalgia, said method comprising the steps of: administering to the patient a dosage form comprising about 12.5 mg, about 25 mg, about 50 mg, or about 100 mg milnacipran or a pharmaceutically acceptable salt thereof (e.g., milnacipran hydrochloride); and providing to the patient prescribing information that comprises dosage, administration, contraindication and/or adverse reaction information pertaining to milnacipran or a pharmaceutically acceptable salt thereof (e.g., milnacipran hydrochloride). In some examples, the contraindication information comprises information indicating that milnacipran or a pharmaceutically acceptable salt thereof (e.g., milnacipran hydrochloride) is contraindicated for humans also taking monoamine oxidase inhibitors. In other examples, the adverse reaction information comprises information indicating that hyperthermia, rigidity, myoclonus, autonomic instability, and/or mental status changes may occur after administering the milnacipran or pharmaceutically acceptable salt thereof (e.g., milnacipran hydrochloride) to the patient. In this regard, the providing of prescribing information bears a strong functional relationship with the successful management of fibromyalgia in patients, due, for example, to increased awareness, understanding, and knowledge by the patient of the proper dosage, regimen, route of administration, usage, and similar information that is achieved by the patients as a result of the prescribing information.

In further embodiments, the present invention provides methods of reducing medication error and enhancing therapeutic compliance in patients diagnosed with fibromyalgia, said method comprising the steps of: administering to the patient a dosage form comprising about 12.5 mg, about 25 mg, about 50 mg, or about 100 mg of milnacipran or a pharmaceutically acceptable salt thereof (e.g., milnacipran hydrochloride); and providing to the patient prescribing information that comprises dosage, administration, contraindication, and adverse reaction information pertaining to milnacipran or a pharmaceutically acceptable salt thereof (e.g., milnacipran hydrochloride). For example, the contraindication information comprises information indicating that milnacipran or a pharmaceutically acceptable salt thereof (e.g., milnacipran hydrochloride) is contraindicated for humans also taking monoamine oxidase inhibitors. In other examples, the adverse reaction information comprises information indicating that hyperthermia, rigidity, myoclonus, autonomic instability, and/or mental status changes may occur after administering the milnacipran or pharmaceutically acceptable salt thereof (e.g., milnacipran hydrochloride) to the patient. Reductions in medication error and enhancements in therapeutic compliance can be assessed and/or quantified in any suitable manner known to those of ordinary skill in the art, and can be readily-ascertained by trained medical professionals, such as by assessment of a decreased incidence of improper patient usage, decreased incidence of side effects brought on by improper usage, and/or any indicia of improved or enhanced symptom relief of fibromyalgia relative to placebo treatment. In this regard, the providing of prescribing information bears a strong functional relationship with assessed reductions in medication error and enhancements in therapeutic compliance in patients, due, for example, to increased awareness, understanding, and knowledge by the patient of the proper dosage, regimen, route of administration, usage, and similar information that is achieved by the patients as a result of the prescribing information.

In a further embodiment, the present invention relates to methods for enhancing patient compliance with a regimen of changing dosage of milnacipran or a pharmaceutically acceptable salt thereof (e.g., milnacipran hydrochloride) over a period of time in a patient diagnosed with fibromyalgia, comprising administering to the patient a titration pack according to any of the embodiments described above.

In certain embodiments, dosage adjustment of milnacipran or a pharmaceutically acceptable salt thereof (e.g., milnacipran hydrochloride) is not necessary in patients with mild renal impairment.

In certain embodiments, milnacipran or a pharmaceutically acceptable salt thereof (e.g., milnacipran hydrochloride) should be used with caution in patients with moderate renal impairment.

In certain embodiments, for patients with severe renal impairment (indicated by an estimated creatinine clearance of 5-29 mL/min), the maintenance dose should be reduced by 50% to about 50 mg/day (about 25 mg twice daily).

In certain embodiments, based on individual patient response, the dose may be increased to about 100 mg/day (about 50 mg twice daily).

In certain embodiments, milnacipran or a pharmaceutically acceptable salt thereof (e.g., milnacipran hydrochloride) is not recommended for patients with end-stage renal disease.

In certain embodiments, dosage adjustment of milnacipran or a pharmaceutically acceptable salt thereof (e.g., milnacipran hydrochloride) is not necessary for patients with hepatic impairment.

In certain embodiments, caution should be exercised in administration of milnacipran or a pharmaceutically acceptable salt thereof (e.g., milnacipran hydrochloride) in patients with severe hepatic impairment.

Withdrawal symptoms have been observed in clinical trials following discontinuation of milnacipran, as with other serotonin and norepinephrine re-uptake inhibitors (SNRIs) and selective serotonin re-uptake inhibitors (SSRIs). In certain embodiments, a patient should be monitored for these symptoms when discontinuing treatment. In further embodiments, the administration of milnacipran or a pharmaceutically acceptable salt thereof (e.g., milnacipran hydrochloride) should be tapered and not abruptly discontinued after extended use.

In certain embodiments, at least 14 days should elapse between discontinuation of a MAOI and initiation of therapy with milnacipran or a pharmaceutically acceptable salt thereof (e.g., milnacipran hydrochloride). In another embodiment, at least 5 days should be allowed after stopping administration milnacipran or a pharmaceutically acceptable salt thereof (e.g., milnacipran hydrochloride) before starting administration of an MAOI.

In further embodiments, film-coated, immediate release tablets in four strengths: 12.5 mg, 25 mg, 50 mg, and 100 mg of milnacipran hydrochloride are provided. For example, 12.5 mg tablets are round, blue, “F” on one side, “L” on the reverse. For example, 25 mg tablets are round, white, “FL” on one side, “25” on the reverse. For example, 50 mg tablets are oval, white, “FL” on one side, “50” on the reverse. For example, 100 mg tablets are oval, pink, “FL” on one side, “100” on the reverse.

In exemplary embodiments, concomitant use of milnacipran hydrochloride in patients taking monoamine oxidase inhibitors (MAOIs) is contraindicated. In patients receiving a serotonin reuptake inhibitor in combination with a monoamine oxidase inhibitor (MAOI), there have been reports of serious, sometimes fatal, reactions including hyperthermia, rigidity, myoclonus, autonomic instability with possible rapid fluctuations of vital signs, and mental status changes that include extreme agitation progressing to delirium and coma. These reactions have also been reported in patients who have recently discontinued serotonin reuptake inhibitors and have been started on an MAOI. Some cases presented with features resembling neuroleptic malignant syndrome. The effects of combined use of milnacipran hydrochloride and MAOIs have not been evaluated in humans. Therefore, in certain embodiments, it is recommended that milnacipran hydrochloride should not be used in combination with an MAOI, or within 14 days of discontinuing treatment with an MAOI. Similarly, at least 5 days should be allowed after stopping milnacipran hydrochloride before starting an MAOI.

In clinical trials, milnacipran hydrochloride was associated with an increased risk of mydriasis. Mydriasis has been reported with other dual reuptake inhibitors of norepinephrine and serotonin. Therefore, in certain embodiments, milnacipran hydrochloride is not administered to patients with uncontrolled narrow-angle glaucoma.

Milnacipran hydrochloride is a selective serotonin and norepinephrine re-uptake inhibitor (SNRI), similar to some drugs used for the treatment of depression and other psychiatric disorders.

Patients, both adult and pediatric, with depression or other psychiatric disorders may experience worsening of their depression and/or the emergence of suicidal ideation and behavior (suicidality) or unusual changes in behavior, whether or not they are taking these medications, and this risk may persist until significant remission occurs. Suicide is a known risk of depression and certain other psychiatric disorders, and these disorders themselves are the strongest predictors of suicide. There has been a long-standing concern, however, that antidepressants, including drugs that inhibit the reuptake of norepinephrine and/or serotonin, may have a role in inducing worsening of depression and the emergence of suicidality in certain patients during the early phases of treatment.

In the placebo-controlled clinical trials of adults with fibromyalgia, among the patients who had a history of depression at treatment initiation, the incidence of suicidal ideation was 0.5% in patients treated with placebo, 0% in patients treated with milnacipran hydrochloride 100 mg/day, and 1.3% in patients treated with milnacipran hydrochloride 200 mg/day. No suicides occurred in the short-term or longer-term (up to 1 year) fibromyalgia trials.

Pooled analyses of short-term placebo-controlled trials of drugs used to treat depression (SSRIs and others) showed that these drugs increase the risk of suicidal thinking and behavior (suicidality) in children, adolescents, and young adults (ages 18-24) with major depressive disorder (MDD) and other psychiatric disorders. Short-term studies did not show an increase in the risk of suicidality with these drugs compared to placebo in adults beyond age 24; there was a reduction in suicidality risk with antidepressants compared to placebo in adults age 65 and older.

The pooled analyses of placebo-controlled trials in children and adolescents with MDD, obsessive compulsive disorder (OCD), or other psychiatric disorders included a total of 24 short-term trials of 9 drugs used to treat depression in over 4400 patients. The pooled analyses of placebo-controlled trials in adults with MDD or other psychiatric disorders included a total of 295 short-term trials (median duration of 2 months) of 11 antidepressant drugs in over 77,000 patients.

There was considerable variation in risk of suicidality among drugs, but a tendency toward an increase in the younger patients for almost all drugs studied. There were differences in absolute risk of suicidality across the different indications, with the highest incidence in MDD. The risk of differences (drug versus placebo), however, were relatively stable within age strata and across indications. These risk differences (drug-placebo difference in the number of cases of suicidality per 1000 patients treated) are provided in Table 1.

TABLE 1
Risk Differences (Drug - Placebo) in the number of
Cases of Suicidality, per 1000 patients treated

| Age Range | |
|---|---|
| | Drug-Placebo Difference in Number of Cases |
| | of Suicidality per 1000 Patients Treated |
| <18 | 14 additional cases |
| 18-24 | 5 additional cases |
| | Decreases Compared to Placebo |
| 25-64 | 1 fewer case |
| ≧65 | 6 fewer cases |

No suicides occurred in any of the pediatric trials. There were suicides in the adult trials, but the number was not sufficient to reach any conclusion about drug effect on suicide.

It is unknown whether the suicidality risk extends to longer-term use, i.e., beyond several months. However, there is substantial evidence from placebo-controlled maintenance trials in adults with depression that the use of antidepressants can delay the recurrence of depression.

All patients being treated with drugs inhibiting the reuptake of norepinephrine and/or serotonin for any indication should be monitored appropriately and observed closely for clinical worsening, suicidality, and unusual changes in behavior, especially during the initial few months of a course of drug therapy, or at times of dose changes, either increases or decreases.

The following symptoms, anxiety, agitation, panic attacks, insomnia, irritability, hostility, aggressiveness, impulsivity, akathisia (psychomotor restlessness), hypomania, mania, have been reported in adult and pediatric patients being treated with drugs inhibiting the reuptake of norepinephrine and/or serotonin for major depressive disorder as well as for other indications, both psychiatric and nonpsychiatric. Although a causal link between the emergence of such symptoms and either the worsening of depression and/or the emergence of suicidal impulses has not been established, there is concern that such symptoms may represent precursors to emerging suicidality.

Consideration should be given to changing the therapeutic regimen, including possibly discontinuing the medication, in patients who may experience worsening depressive symptoms, or who are experiencing emergent suicidality or symptoms that might be precursors to worsening depression or suicidality, especially if these symptoms are severe or abrupt in onset, or were not part of the patient's presenting symptoms.

If the decision has been made to discontinue treatment due to worsening depressive symptoms or emergent suicidality, medication should be tapered, as rapidly as is feasible, but with recognition that abrupt discontinuation can produce withdrawal symptoms.

Families and caregivers of patients being treated with drugs inhibiting the reuptake of norepinephrine and/or serotonin for major depressive disorder or other indications, both psychiatric and nonpsychiatric, should be alerted about the need to monitor patients for the emergence of agitation, irritability, unusual changes in behavior, and the other symptoms described above, as well as the emergence of suicidality, and to report such symptoms immediately to health care providers. Such monitoring should include daily observation by families and caregivers. In certain embodiments, prescriptions for milnacipran hydrochloride should be written for the smallest quantity of tablets consistent with good patient management, in order to reduce the risk of overdose.

The development of a potentially life-threatening serotonin syndrome may occur with agents that inhibit serotonin reuptake, including milnacipran hydrochloride particularly with concomitant use of serotonergic drugs (including triptans and tramadol) and with drugs which impair metabolism of serotonin (including MAOIs). Serotonin syndrome symptoms may include mental status changes (e.g., agitation, hallucinations, coma), autonomic instability (e.g., tachycardia, labile blood pressure, hyperthermia), neuromuscular aberrations (e.g., hyperreflexia, incoordination) and/or gastrointestinal symptoms (e.g., nausea, vomiting, diarrhea).

In certain embodiments, the concomitant use of milnacipran hydrochloride with MAOIs is contraindicated.

If concomitant treatment of milnacipran hydrochloride with a 5-hydroxytryptamine receptor agonist (triptan) is clinically warranted, careful observation of the patient is advised, particularly during treatment initiation and dose increases.

In certain embodiments, the concomitant use of milnacipran hydrochloride with serotonin precursors (such as tryptophan) is not recommended.

Inhibition of the reuptake of norepinephrine (NE) and serotonin (5-HT) can lead to cardiovascular effects. SNRIs, including milnacipran hydrochloride have been associated with reports of increase in blood pressure.

In a double-blind, placebo-controlled clinical pharmacology study in healthy subjects designed to evaluate the effects of milnacipran on various parameters, including blood pressure at supratherapeutic doses, there was evidence of mean increases in supine blood pressure at doses up to 300 mg twice daily (600 mg/day). At the highest 300 mg twice daily dose, the mean increase in systolic blood pressure was up to 8.1 mm Hg for the placebo group and up to 10.0 mm Hg for the milnacipran hydrochloride treated group over the 12 hour steady state dosing interval. The corresponding mean increase in diastolic blood pressure over this interval was up to 4.6 mm Hg for placebo and up to 11.5 mm Hg for the milnacipran hydrochloride treated group.

In the 3-month placebo-controlled fibromyalgia clinical trials, milnacipran hydrochloride treatment was associated with mean increases of up to 3.1 mm Hg in systolic blood pressure (SBP) and diastolic blood pressure (DBP).

In the placebo-controlled trials, among fibromyalgia patients who were non-hypertensive at baseline, approximately twice as many patients in the milnacipran hydrochloride treatment arms became hypertensive at the end of the study (SBP≧140 mmHg or DBP≧90 mmHg) compared with the placebo patients: 7.2% of patients in the placebo arm versus 19.5% of patients treated with milnacipran hydrochloride 100 mg/day and 16.6% of patients treated with milnacipran hydrochloride 200 mg/day. Among patients who met systolic criteria for pre-hypertension at baseline (SBP 120-139 mmHg), more patients became hypertensive at the end of the study in the milnacipran hydrochloride treatment arms than placebo: 9% of patients in the placebo arm versus 14% in both the milnacipran hydrochloride 100 mg/day and the milnacipran hydrochloride 200 mg/day treatment arms.

Among fibromyalgia patients who were hypertensive at baseline, more patients in the milnacipran hydrochloride treatment arms had a >15 mmHg increase in SBP than placebo at the end of the study: 1% of patients in the placebo aim versus 7% in the milnacipran hydrochloride 100 mg/day and 2% in the milnacipran hydrochloride 200 mg/day treatment arms. Similarly, more patients who were hypertensive at baseline and were treated with milnacipran hydrochloride had DBP increases >10 mmHg than placebo at the end of study: 3% of patients in the placebo arm versus 8% in the milnacipran hydrochloride 100 mg/day and 6% in the milnacipran hydrochloride 200 mg/day treatment arms.

Sustained increases in SBP (increase of ≧15 mmHg on three consecutive post-baseline visits) occurred in 2% of placebo patients versus 9% of patients receiving milnacipran hydrochloride 100 mg/day and 6% of patients receiving milnacipran hydrochloride 200 mg/day. Sustained increases in DBP (increase of ≧10 mmHg on 3 consecutive post-baseline visits) occurred in 4% of patients receiving placebo versus 13% of patients receiving milnacipran hydrochloride 100 mg/day and 10% of patients receiving milnacipran hydrochloride 200 mg/day.

Sustained increases in blood pressure could have adverse consequences. Cases of elevated blood pressure requiring immediate treatment have been reported.

Concomitant use of milnacipran hydrochloride with drugs that increase blood pressure and pulse has not been evaluated and such combinations should be used with caution. In certain embodiments, milnacipran hydrochloride should not be use din conjunction with a drug that increases blood pressure and/or pulse.

Effects of milnacipran hydrochloride on blood pressure in patients with significant hypertension or cardiac disease have not been systematically evaluated. In certain embodiments, milnacipran hydrochloride should be used with caution in patients with significant hypertension or cardiac disease.

Blood pressure should be measured prior to initiating treatment and periodically measured throughout milnacipran hydrochloride treatment. Pre-existing hypertension and other cardiovascular disease should be treated before starting therapy with milnacipran hydrochloride. For patients who experience a sustained increase in blood pressure while receiving milnacipran hydrochloride, either dose reduction or discontinuation should be considered.

SNRIs have been associated with reports of increase in heart rate.

In clinical trials, relative to placebo, milnacipran hydrochloride treatment was associated with mean increases in pulse rate of approximately 7 to 8 beats per minute.

Increases in pulse≧20 bpm occurred more frequently in milnacipran hydrochloride treated patients when compared to placebo: 0.3% in the placebo arm versus 8% in the milnacipran hydrochloride 100 mg/day and 8% in the 200 mg/day treatment arms. The effect of milnacipran hydrochloride on heart rate did not appear to increase with increasing dose.

Milnacipran hydrochloride has not been systematically evaluated in patients with a cardiac rhythm disorder.

Heart rate should be measured prior to initiating treatment and periodically measured throughout milnacipran hydrochloride treatment. Pre-existing tachyarrhythmias and other cardiac disease should be treated before starting therapy with milnacipran hydrochloride In certain embodiments, for patients who experience a sustained increase in heart rate while receiving milnacipran hydrochloride either dose reduction or discontinuation should be considered.

Milnacipran hydrochloride has not been systematically evaluated in patients with a seizure disorder. In clinical trials evaluating milnacipran hydrochloride in patients with fibromyalgia, seizures/convulsions have not been reported. However, seizures have been reported infrequently in patients treated with milnacipran hydrochloride for disorders other than fibromyalgia. In certain embodiments, milnacipran hydrochloride should be prescribed with care in patients with a history of a seizure disorder.

In the placebo-controlled fibromyalgia trials, increases in the number of patients treated with milnacipran hydrochloride with mild elevations of ALT or AST (1-3 times the upper limit of normal, ULN) were observed. Increases in ALT were more frequently observed in the patients treated with milnacipran hydrochloride 100 mg/day (6%) and milnacipran hydrochloride 200 mg/day (7%), compared to the patients treated with placebo (3%). One patient receiving milnacipran hydrochloride 100 mg/day (0.2%) had an increase in ALT greater than 5 times the upper limit of normal but did not exceed 10 times the upper limit of normal. Increases in AST were more frequently observed in the patients treated with milnacipran hydrochloride 100 mg/day (3%) and milnacipran hydrochloride 200 mg/day (5%) compared to the patients treated with placebo (2%).

The increases of bilirubin observed in the fibromyalgia clinical trials were not clinically significant.

No case met the criteria of elevated ALT>3×ULN and associated with an increase in bilirubin≧2×ULN.

There have been cases of increased liver enzymes and reports of severe liver injury, including fulminant hepatitis with milnacipran from foreign postmarketing experience. In the cases of severe liver injury there were significant underlying clinical conditions and/or the use of multiple concomitant medications. Because of underreporting, it is impossible to provide an accurate estimate of the true incidence of these reactions.

Milnacipran hydrochloride should be discontinued in patients who develop jaundice or other evidence of liver dysfunction. Treatment with milnacipran hydrochloride should not be resumed unless another cause can be established.

In certain embodiments, milnacipran hydrochloride should ordinarily not be prescribed to patients with substantial alcohol use or evidence of chronic liver disease.

Withdrawal symptoms have been observed in clinical trials following discontinuation of milnacipran, as with other SNRIs and SSRIs.

During marketing of milnacipran, and other SNRIs and SSRIs, there have been spontaneous reports of adverse events indicative of withdrawal and physical dependence occurring upon discontinuation of these drugs, particularly when discontinuation is abrupt. The adverse events include the following: dysphoric mood, irritability, agitation, dizziness, sensory disturbances (e.g., paresthesias such as electric shock sensations), anxiety, confusion, headache, lethargy, emotional lability, insomnia, hypomania, tinnitus, and seizures. Although these events are generally self-limiting, some have been reported to be severe.

Patients should be monitored for these symptoms when discontinuing treatment with milnacipran hydrochloride. In certain embodiments, milnacipran hydrochloride should be tapered and not abruptly discontinued after extended use. If intolerable symptoms occur following a decrease in the dose or upon discontinuation of treatment, then in certain embodiments, resuming the previously prescribed dose may be considered. Subsequently, the physician may continue decreasing the dose but at a more gradual rate.

Hyponatremia may occur as a result of treatment with SSRIs and SNRIs, including milnacipran hydrochloride. In many cases, this hyponatremia appears to be the result of the syndrome of inappropriate antidiuretic hormone secretion (SIADH). Cases with serum sodium lower than 110 mmol/L have been reported. Elderly patients may be at greater risk of developing hyponatremia with SNRIs, SSRIs, or milnacipran hydrochloride Also, patients taking diuretics or who are otherwise volume-depleted may be at greater risk. In certain embodiments, discontinuation of milnacipran hydrochloride should be considered in patients with symptomatic hyponatremia.

Signs and symptoms of hyponatremia include headache, difficulty concentrating, memory impairment, confusion, weakness, and unsteadiness, which may lead to falls. Signs and symptoms associated with more severe and/or acute cases have included hallucination, syncope, seizure, coma, respiratory arrest, and death.

SSRIs and SNRIs, including milnacipran hydrochloride may increase the risk of bleeding events. Concomitant use of aspirin, nonsteroidal anti-inflammatory drugs, warfarin, and other anti-coagulants may add to this risk. Case reports and epidemiological studies (case-control and cohort design) have demonstrated an association between use of drugs that interfere with serotonin reuptake and the occurrence of gastrointestinal bleeding. Bleeding events related to SSRIs and SNRIs use have ranged from ecchymoses, hematomas, epistaxis, and petechiae to life-threatening hemorrhages.

In certain embodiments, patients should be cautioned about the risk of bleeding associated with the concomitant use of milnacipran hydrochloride and NSAIDs, aspirin, or other drugs that affect coagulation.

No activation of mania or hypomania was reported in the clinical trials evaluating effects of milnacipran hydrochloride in patients with fibromyalgia. However those clinical trials excluded patients with current major depressive episode. Activation of mania and hypomania have been reported in patients with mood disorders who were treated with other similar drugs for major depressive disorder. As with these other agents, in additional embodiments, milnacipran hydrochloride should be used cautiously in patients with a history of mania.

Because of their noradrenergic effect, SNRIs including milnacipran hydrochloride can affect urethral resistance and micturition. In the controlled fibromyalgia trials, dysuria occurred more frequently in patients treated with milnacipran hydrochloride (1%) than in placebo-treated patients (0.5%). IN certain embodiments, caution is advised in use of milnacipran hydrochloride in patients with a history of dysuria, notably in male patients with prostatic hypertrophy, prostatitis, and other lower urinary tract obstructive disorders. Male patients are more prone to genitourinary adverse effects, such as dysuria or urinary retention, and may experience testicular pain or ejaculation disorders.

Mydriasis has been reported in association with SNRIs and milnacipran hydrochloride. In additional embodiments, milnacipran hydrochloride should be used cautiously in patients with controlled narrow-angle glaucoma.

In further embodiments milnacipran hydrochloride is not administered to patients with Uncontrolled Narrow-Angle Glaucoma.

In clinical trials, more patients treated with milnacipran hydrochloride developed elevated transaminases than did placebo treated patients. Because it is possible that milnacipran may aggravate pre-existing liver disease, in additional embodiments, milnacipran hydrochloride should not be prescribed to patients with substantial alcohol use or evidence of chronic liver disease.

Milnacipran hydrochloride was evaluated in three double-blind placebo-controlled trials involving 2209 fibromyalgia patients (1557 patients treated with milnacipran hydrochloride and 652 patients treated with placebo) for a treatment period up to 29 weeks.

The stated frequencies of adverse reactions represent the proportion of individuals who experienced, at least once, a treatment-emergent adverse reaction of the type listed. A reaction was considered treatment emergent if it occurred for the first time or worsened while receiving therapy following baseline evaluation.

Because clinical trials are conducted under widely varying conditions, adverse reaction rates observed in the clinical trials of a drug cannot be directly compared to rates in the clinical trials of another drug and may not reflect the rates observed in practice.

In placebo-controlled trials in patients with fibromyalgia, 23% of patients treated with milnacipran hydrochloride 100 mg/day, 26% of patients treated with milnacipran hydrochloride 200 mg/day discontinued prematurely due to adverse reactions, compared to 12% of patients treated with placebo. The adverse reactions that led to withdrawal in ≧1% of patients in the milnacipran hydrochloride treatment group and with an incidence rate greater than that in the placebo treatment group were nausea (milnacipran 6%, placebo 1%), palpitations (milnacipran 3%, placebo 1%), headache (milnacipran 2%, placebo 0%), constipation (milnacipran 1%, placebo 0%), heart rate increased (milnacipran 1%, placebo 0%), and hyperhidrosis (milnacipran 1%, placebo 0%), vomiting (milnacipran 1%, placebo 0%), and dizziness (milnacipran 1% and placebo 0.5%). Discontinuation due to adverse reactions was generally more common among patients treated with milnacipran hydrochloride 200 mg/day compared to milnacipran hydrochloride 100 mg/day.

In the placebo-controlled fibromyalgia patient trials the most frequently occurring adverse reaction in clinical trials was nausea. The most common adverse reactions (incidence≧5% and twice placebo) in patients treated with milnacipran hydrochloride were constipation, hot flush, hyperhidrosis, vomiting, palpitations, heart rate increased, dry mouth, and hypertension.

Table 2 lists all adverse reactions that occurred in at least 2% of patients treated with milnacipran hydrochloride at either 100 or 200 mg/day and at an incidence greater than that of placebo.

TABLE 2
Treatment-Emergent Adverse Reaction Incidence in Placebo Controlled Trials
in Fibromyalgia Patients (Events Occurring in at Least 2% of All milnacipran
hydrochloride Treated Patients and Occurring More Frequently in Either milnacipran
hydrochloride Treatment Group Than in the Placebo Treatment Group)

| | Milnacipran | Milnacipran | All | |
|---|---|---|---|---|
| | Hydrochloride | Hydrochloride | Milnacipran | |
| System Organ Class- | 100 mg/day | 200 mg/day | Hydrochloride | Placebo |
| Preferred Term | (n = 623) % | (n = 934) % | (n = 1557) % | (n = 652) % |
| Cardiac Disorders | | | | |
| Palpitations | 8 | 7 | 7 | 2 |
| Tachycardia | 3 | 2 | 2 | 1 |
| Eye Disorders | | | | |
| Vision blurred | 1 | 2 | 2 | 1 |
| Gastrointestinal Disorders | | | | |
| Nausea | 35 | 39 | 37 | 20 |
| Constipation | 16 | 15 | 16 | 4 |
| Vomiting | 6 | 7 | 7 | 2 |
| Dry mouth | 5 | 5 | 5 | 2 |
| Abdominal pain | 3 | 3 | 3 | 2 |
| General Disorders | | | | |
| Chest pain | 3 | 2 | 2 | 2 |
| Chills | 1 | 2 | 2 | 0 |
| Chest discomfort | 2 | 1 | 1 | 1 |
| Infections | | | | |
| Upper respiratory tract | 7 | 6 | 6 | 6 |
| infection | | | | |
| Investigations | | | | |
| Heart rate increased | 5 | 6 | 6 | 1 |
| Blood pressure increased | 3 | 3 | 3 | 1 |
| Metabolism and Nutrition | | | | |
| Disorders | | | | |
| Decreased appetite | 1 | 2 | 2 | 0 |
| Nervous System Disorders | | | | |
| Headache | 19 | 17 | 18 | 14 |
| Dizziness | 11 | 10 | 10 | 6 |
| Migraine | 6 | 4 | 5 | 3 |
| Paresthesia | 2 | 3 | 2 | 2 |
| Tremor | 2 | 2 | 2 | 1 |
| Hypoesthesia | 1 | 2 | 1 | 1 |
| Tension headache | 2 | 1 | 1 | 1 |
| Psychiatric Disorders | | | | |
| Insomnia | 12 | 12 | 12 | 10 |
| Anxiety | 5 | 3 | 4 | 4 |
| Respiratory Disorders | | | | |
| Dyspnea | 2 | 2 | 2 | 1 |
| Skin Disorders | | | | |
| Hyperhidrosis | 8 | 9 | 9 | 2 |
| Rash | 3 | 4 | 3 | 2 |
| Pruritus | 3 | 2 | 2 | 2 |
| Vascular Disorders | | | | |
| Hot flush | 11 | 12 | 12 | 2 |
| Hypertension | 7 | 4 | 5 | 2 |
| Flushing | 2 | 3 | 3 | 1 |

In placebo-controlled fibromyalgia clinical trials, patients treated with milnacipran hydrochloride for up to 3 months experienced a mean weight loss of approximately 0.8 kg in both the milnacipran hydrochloride 100 mg/day and the milnacipran hydrochloride 200 mg/day treatment groups, compared with a mean weight loss of approximately 0.2 kg in placebo-treated patients.

In the placebo-controlled fibromyalgia studies, the following treatment-emergent adverse reactions related to the genitourinary system were observed in at least 2% of male patients treated with milnacipran hydrochloride and occurred at a rate greater than in placebo-treated male patients: dysuria, ejaculation disorder, erectile dysfunction, ejaculation failure, libido decreased, prostatitis, scrotal pain, testicular pain, testicular swelling, urinary hesitation, urinary retention, urethral pain, and urine flow decreased.

The following is a list of frequent (those occurring on one or more occasions in at least 1/100 patients) treatment-emergent adverse reactions reported from 1824 fibromyalgia patients treated with milnacipran hydrochloride for periods up to 68 weeks. The listing does not include those events already listed in Table 1, those events for which a drug cause was remote, those events which were so general as to be uninformative, and those events reported only once which did not have a substantial probability of being acutely life threatening.

Adverse reactions are categorized by body system and listed in order of decreasing frequency. Adverse reactions of major clinical importance are described above.

Gastrointestinal Disorders: diarrhea, dyspepsia, gastroesophageal reflux disease, flatulence, abdominal distension

General Disorders: fatigue, peripheral edema, irritability, pyrexia

Infections: urinary tract infection, cystitis

Injury, Poisoning, and Procedural Complications: contusion, fall

Investigations: weight decreased or increased

Metabolism and Nutrition Disorders: hypercholesterolemia

Nervous System Disorders: somnolence, dysgeusia

Psychiatric Disorders: depression, stress

Skin Disorders: night sweats

The following additional adverse reactions have been identified from spontaneous reports of milnacipran hydrochloride received worldwide. These adverse reactions have been chosen for inclusion because of a combination of seriousness, frequency of reporting, or potential causal connection to milnacipran hydrochloride. However, because these adverse reactions were reported voluntarily from a population of uncertain size, it is not always possible to reliably estimate their frequency or establish a causal relationship to drug exposure. These events include:

Blood and Lymphatic System Disorders: leukopenia, neutropenia, thrombocytopenia

Cardiac Disorders: supraventricular tachycardia

Eye Disorders: accommodation disorder

Endocrine Disorders: hyperprolactinemia

Hepatobiliary Disorders: hepatitis

Metabolism and Nutrition Disorders: anorexia, hyponatremia

Musculoskeletal and Connective Tissue Disorders: rhabdomyolysis

Nervous System Disorders: convulsions (including grand mal), loss of consciousness, neuroleptic malignant syndrome, Parkinsonism, serotonin syndrome

Psychiatric Disorders: delirium, hallucination

Renal and Urinary Disorders: acute renal failure

Reproductive System and Breast Disorders: galactorrhea

Skin Disorders: erythema multiforme, Stevens Johnson syndrome

Vascular Disorders: hypertensive crisis

Milnacipran undergoes minimal CYP450 related metabolism, with the majority of the dose excreted unchanged in urine (55%), and has a low binding to plasma proteins (13%). In vitro and in vivo studies showed that milnacipran hydrochloride is unlikely to be involved in clinically significant pharmacokinetic drug interactions

Serotonin syndrome may occur when lithium is co-administered with milnacipran hydrochloride and with other drugs that impair metabolism of serotonin.

Milnacipran hydrochloride inhibits the reuptake of norepinephrine. Therefore concomitant use of milnacipran hydrochloride with epinephrine and norepinephrine may be associated with paroxysmal hypertension and possible arrhythmia. In certain embodiments, milnacipran hydrochloride is not co-administered with epinephrine and/or norepinephrine.

Co-administration of milnacipran hydrochloride with other inhibitors of serotonin re-uptake may result in hypertension and coronary artery vasoconstriction, through additive serotonergic effects.

Use of milnacipran hydrochloride concomitantly with digoxin may be associated with potentiation of adverse hemodynamic effects. Postural hypotension and tachycardia have been reported in combination therapy with intravenously administered digoxin (1 mg). Co-administration of milnacipran hydrochloride and intravenous digoxin should be avoided. In certain embodiments, milnacipran hydrochloride is not co-administered with digoxin.

Because milnacipran hydrochloride inhibits norepinephrine reuptake, co-administration with clonidine may inhibit clonidine's anti-hypertensive effect. In certain embodiments, milnacipran hydrochloride is not co-administered with clonidine.

In a drug-drug interaction study, an increase in euphoria and postural hypotension was observed in patients who switched from clomipramine to milnacipran hydrochloride.

Given the primary CNS effects of milnacipran hydrochloride, caution should be used when it is taken in combination with other centrally acting drugs, including those with a similar mechanism of action.

### Pregnancy Category C

Milnacipran increased the incidence of dead fetuses in utero in rats at doses of 5 mg/kg/day (0.25 times the MRHD on a mg/m² basis). Administration of milnacipran to mice and rabbits during the period of organogenesis did not result in embryotoxicity or teratogenicity at doses up to 125 mg/kg/day in mice (3 times the maximum recommended human dose [MRHD] of 200 mg/day on a mg/m² basis) and up to 60 mg/kg/day in rabbits (6 times the MRHD of 200 mg/day on a mg m² basis). In rabbits, the incidence of the skeletal variation, extra single rib, was increased following administration of milnacipran at 15 mg/kg/day during the period of organogenesis.

There are no adequate and well-controlled studies in pregnant women. Milnacipran hydrochloride should be used during pregnancy only if the potential benefit justifies the potential risk to the fetus.

Neonates exposed to dual reuptake inhibitors of serotonin and norepinephrine, or selective serotonin reuptake inhibitors late in the third trimester have developed complications requiring prolonged hospitalization, respiratory support, and tube feeding. Such complications can arise immediately upon delivery. Reported clinical findings have included respiratory distress, cyanosis, apnea, seizures, temperature instability, feeding difficulty, vomiting, hypoglycemia, hypotonia, hypertonia, hyperreflexia, tremor, jitteriness, irritability, and constant crying. These features are consistent with either a direct toxic effect of these classes of drugs or, possibly, a drug discontinuation syndrome. It should be noted that, in some cases, the clinical picture is consistent with serotonin syndrome.

In rats, a decrease in pup body weight and viability on postpartum day 4 were observed when milnacipran, at a dose of 5 mg/kg/day (approximately 0.2 times the MRHD on a mg/m² basis), was administered orally to rats during late gestation. The no-effect dose for maternal and offspring toxicity was 2.5 mg/kg/day (approximately 0.1 times the MRHD on a mg/m² basis).

The effect of milnacipran on labor and delivery is unknown. The use of milnacipran hydrochloride during labor and delivery is not recommended. In certain embodiments, milnacipran hydrochloride is not administered during labor and delivery.

There are no adequate and well-controlled studies in nursing mothers. It is not known if milnacipran is excreted in human milk. Studies in animals have shown that milnacipran or its metabolites are excreted in breast milk. Because many drugs are excreted in human milk and because of the potential for serious adverse reactions in nursing infants from milnacipran, a decision should be made whether to discontinue the drug, taking into account the importance of the drug to the mother. Because the safety of milnacipran hydrochloride in infants is not known, nursing while on milnacipran hydrochloride is not recommended. In certain embodiments, milnacipran hydrochloride is not administered to patients who are nursing.

Safety and effectiveness of milnacipran hydrochloride in a fibromyalgia pediatric population below the age of 17 have not been established. The use of milnacipran hydrochloride is not recommended in pediatric patients. In certain embodiments, milnacipran hydrochloride is not administered to pediatric patients

In controlled clinical studies of milnacipran hydrochloride, 402 patients were 60 years or older, and no overall differences in safety and efficacy were observed between these patients and younger patients.

In view of the predominant excretion of unchanged milnacipran via kidneys and the expected decrease in renal function with age renal function should be considered prior to use of milnacipran hydrochloride in the elderly.

SNRIs, SSRIs, and milnacipran hydrochloride have been associated with cases of clinically significant hyponatremia in elderly patients, who may be at greater risk for this adverse event.

Milnacipran is not a controlled substance. Milnacipran did not produce behavioral signs indicative of abuse potential in animal or human studies.

Milnacipran produces physical dependence, as evidenced by the emergence of withdrawal symptoms following drug discontinuation, similar to other SNRIs and SSRIs. These withdrawal symptoms can be severe. Thus, in certain embodiments, milnacipran hydrochloride should be tapered and not abruptly discontinued after extended use.

There is limited clinical experience with milnacipran hydrochloride overdose in humans. In clinical trials, cases of acute ingestions up to 1000 mg, alone or in combination with other drugs, were reported with none being fatal.

In post-marketing experience, fatal outcomes have been reported for acute overdoses primarily involving multiple drugs but also with milnacipran hydrochloride only. The most common signs and symptoms included increased blood pressure, cardio-respiratory arrest, changes in the level of consciousness (ranging from somnolence to coma), confusional state, dizziness, and increased hepatic enzymes.

There is no specific antidote to milnacipran hydrochloride but if serotonin syndrome ensues, specific treatment (such as with cyproheptadine and/or temperature control) may be considered. In case of acute overdose, treatment should consist of those general measures employed in the management of overdose with any drug.

An adequate airway, oxygenation, and ventilation should be assured and cardiac rhythm and vital signs should be monitored. Induction of emesis is not recommended. Gastric lavage with a large-bore orogastric tube with appropriate airway protection, if needed, may be indicated if performed soon after ingestion or in symptomatic patients. Because there is no specific antidote for milnacipran hydrochloride symptomatic care and treatment with gastric lavage and activated charcoal should be considered as soon as possible for patients who experience a milnacipran hydrochloride overdose.

Due to the large volume of distribution of this drug, forced diuresis, dialysis, hemoperfusion, and exchange transfusion are unlikely to be beneficial.

In managing overdose, the possibility of multiple drug involvement should be considered. The physician should consider contacting a poison control center for additional information on the treatment of any overdose. Telephone numbers for certified poison control centers are listed in the Physicians' Desk Reference (PDR).

The exact mechanism of the central pain inhibitory action of milnacipran and its ability to improve the symptoms of fibromyalgia in humans are unknown. Preclinical studies have shown that milnacipran is a potent inhibitor of neuronal norepinephrine and serotonin reuptake; milnacipran inhibits norepinephrine uptake with approximately 3-fold higher potency in vitro than serotonin without directly affecting the uptake of dopamine or other neurotransmitters. Milnacipran has no significant affinity for serotonergic (5-HT1-7), α- and β-adrenergic, muscarinic (M1-5), histamine (H1-4), dopamine (D1-5), opiate, benzodiazepine, and γ-aminobutyric acid (GABA) receptors in vitro. Pharmacologic activity at these receptors is hypothesized to be associated with the various anticholinergic, sedative, and cardiovascular effects seen with other psychotropic drugs.

Milnacipran has no significant affinity for Ca⁺⁺, K⁺, Na⁺ and Cl⁻ channels and does not inhibit the activity of human monoamine oxidases (MAO-A and MAO-B) or acetylcholinesterase.

Cardiovascular Electrophysiology

The effect of milnacipran hydrochloride on the QTcF interval was measured in a double-blind placebo- and positive-controlled parallel study in 88 healthy subjects using 600 mg/day SAVELLA™ (3 to 6 times the recommended therapeutic dose for fibromyalgia). After baseline and placebo adjustment, the maximum mean QTcF change was 8 ms (2-sided 90% CI, 3-12 ms). This increase is not considered to be clinically significant.

Milnacipran is well absorbed after oral administration with an absolute bioavailability of approximately 85% to 90%. The exposure to milnacipran increased proportionally within the therapeutic dose range. It is excreted predominantly unchanged in urine (55%) and has a terminal elimination half-life of about 6 to 8 hours. Steady-state levels are reached within 36 to 48 hours and can be predicted from single-dose data. The active (1S,2R) enantiomer has a longer elimination half-life (8-10 hours) than the (1R,2S) enantiomer (4-6 hours). There is no interconversion between the enantiomers.

Milnacipran hydrochloride is absorbed following oral administration with maximum concentrations (Cₘₐₓ) reached within 2 to 4 hours post dose. Absorption of milnacipran hydrochloride is not affected by food. The absolute bioavailability is approximately 85% to 90%. The mean volume of distribution of milnacipran following a single intravenous dose to healthy subjects is approximately 400 L. Plasma protein binding is 13%.

Milnacipran and its metabolites are eliminated primarily by renal excretion. Following oral administration of ¹⁴C-milnacipran hydrochloride, approximately 55% of the dose was excreted in urine as unchanged milnacipran (24% as l-milnacipran and 31% as d-milnacipran). The l-milnacipran carbamoyl-O-glucuronide was the major metabolite excreted in urine and accounted for approximately 17% of the dose; approximately 2% of the dose was excreted in urine as d-milnacipran carbamoyl-O-glucuronide. Approximately 8% of the dose was excreted in urine as the N-desethyl milnacipran metabolite.

Milnacipran pharmacokinetics were evaluated following single oral administration of 50 mg milnacipran hydrochloride to subjects with mild (creatinine clearance [CLcr] 50-80 mL/min), moderate (CLcr 30-49 mL/min), and severe (CLcr 5-29 mL/min) renal impairment and to healthy subjects (CLcr>80 mL/min). The mean AUC0-∞ increased by 16%, 52%, and 199%, and terminal elimination half-life increased by 38%, 41%, and 122% in subjects with mild, moderate, and severe renal impairment, respectively, compared with healthy subjects.

No dosage adjustment is necessary for patients with mild renal impairment. Caution should be exercised in patients with moderate renal impairment. Dose adjustment is necessary in severe renal impairment patients.

Milnacipran pharmacokinetics were evaluated following single oral administration of 50 mg Savella to subjects with mild (Child-Pugh A), moderate (Child-Pugh B), and severe (Child-Pugh C) hepatic impairment and to healthy subjects. AUC0-∞ and T½ were similar in healthy subjects and subjects with mild and moderate hepatic impairment. However, subjects with severe hepatic impairment had a 31% higher AUC0-∞ and a 55% higher T½ than healthy subjects. Caution should be exercised in patients with severe hepatic impairment.

Cₘₐₓ and AUC parameters of milnacipran were about 30% higher in elderly (>65 years) subjects compared with young subjects due to age-related decreases in renal function.

No dosage adjustment is necessary based on age unless renal function is severely impaired.

Cₘₐₓ and AUC parameters of milnacipran were about 20% higher in female subjects compared with male subjects. Dosage adjustment based on gender is not necessary.

### In Vitro Studies

In general, milnacipran, at concentrations that were at least 25 times those attained in clinical trials, did not inhibit human CYP1A2, CYP2A6, CYP2C9, CYP2C19, CYP2D6, CYP2E1, and CYP3A4 or induce human CYP1A2, CYP2B6, CYP2C8, CYP2C9, CYP2C19, and CYP3A4/5 enzyme systems, indicating a low potential of interactions with drugs metabolized by these enzymes.

In vitro studies have shown that the biotransformation rate of milnacipran by human hepatic microsomes and hepatocytes was low. A low biotransformation was also observed following incubation of milnacipran with cDNA-expressed human CYP1A2, CYP2A6, CYP2B6, CYP2C9, CYP2C19, CYP2D6, CYP2E1, and CYP3A4 isozymes.

### In Vivo Studies

The drug interaction studies described below were conducted in healthy adult subjects.

### Carbamazepine

There were no clinically significant changes in the pharmacokinetics of milnacipran following coadministration of milnacipran hydrochloride (100 mg/day) and carbamazepine (200 mg twice a day). No changes were observed in the pharmacokinetics of carbamazepine or its epoxide metabolite due to co-administration with milnacipran hydrochloride.

### Clomipramine

Switch from clomipramine (75 mg once a day) to milnacipran (100 mg/day) without a washout period did not lead to clinically significant changes in the pharmacokinetics of milnacipran. Because an increase in adverse events (e.g., euphoria and postural hypotension) was observed after switching from clomipramine to milnacipran, monitoring of patients during treatment switch is recommended.

### Digoxin

There was no pharmacokinetic interaction between milnacipran hydrochloride (200 mg/day) and digoxin (0.2 mg/day Lanoxicaps) following multiple-dose administration to healthy subjects.

### Fluoxetine

Switch from fluoxetine (20 mg once a day), a strong inhibitor of CYP2D6 and a moderate inhibitor of CYP2C19, to milnacipran (100 mg/day) without a washout period did not affect the pharmacokinetics of milnacipran.

### Lithium

Multiple doses of milnacipran hydrochloride (100 mg/day) did not affect the pharmacokinetics of lithium.

### Lorazepam

There was no pharmacokinetic interaction between a single dose of milnacipran hydrochloride (50 mg) and lorazepam (1.5 mg).

### Warfarin

Steady-state milnacipran (200 mg/day) did not affect the pharmacokinetics of R-warfarin and S-warfarin or the pharmacodynamics (as assessed by measurement of prothrombin INR) of a single dose of 25 mg warfarin. The pharmacokinetics of milnacipran hydrochloride were not altered by warfarin.

### Carcinogenesis, Mutagenesis, and Impairment of Fertility

Dietary administration of milnacipran to rats at doses of 50 mg/kg/day (2 times the MRHD on a mg/m² basis) for 2 years caused a statistically significant increase in the incidence of thyroid C-cell adenomas and combined adenomas and carcinomas in males. A carcinogenicity study was conducted in Tg.rasH2 mice for 6 months at oral gavage doses of up to 125 mg/kg/day. Milnacipran did not induce tumors in Tg.rasH2 mice at any dose tested.

Milnacipran was not mutagenic in the in vitro bacterial reverse mutation assay (Ames test) or in the L5178Y TK+/−mouse lymphoma forward mutation assay. Milnacipran was also not clastogenic in an in vitro chromosomal aberration test in human lymphocytes or in the in vivo mouse micronucleus assay.

Although administration of milnacipran to male and female rats had no statistically significant effect on mating or fertility at doses up to 80 mg/kg/day (4 times the MRHD on an mg/m² basis) there was an apparent dose-related decrease in the fertility index at clinically relevant doses based on body surface area.

### Animal Toxicology and Pharmacology

Chronic administration (2-years) of milnacipran to rats at 15 mg/kg (0.6 times the MRHD on an mg/m² basis) and higher doses showed increased incidences of centrilobular vacuolation of the liver in male rats and eosinophilic foci in male and female rats in the absence of any change in hepatic enzymes. The clinical significance of the finding is not known. Chronic (1-year) administration in the primate at doses up to 25 mg/kg (2 times the MRHD on a mg/m² basis) did not demonstrate similar evidence of hepatic changes.

Chronic (2-years) administration of milnacipran to rats at 15 mg/kg (0.6 times the MRHD on a mg/m² basis) and higher doses showed increased incidence of keratitis of the eye. One year studies in the rat and primate did not show this response.

In certain embodiments, milnacipran hydrochloride may be supplied in the following forms:

### 12.5-mg Tablets:

Blue, round, film-coated tablets, debossed with “F” on one side and “L” on the reverse

Bottles of 60: NDC 0456-1512-60

### 25-mg Tablets:

White, round, film-coated tablets, debossed with “FL” on one side and “25” on the reverse

Bottles of 60: NDC 0456-1525-60

Bottles of 180: NDC 0456-1525-01

### 50-mg Tablets:

White, oval-shaped, film-coated tablets, debossed with “FL” on one side and “50” on the reverse

Bottles of 60: NDC 0456-1550-60

Bottles of 180: NDC 0456-1550-01

### 100-mg Tablets:

Pink, oval-shaped film-coated tablets, debossed with “FL” on one side and “100” on the reverse

Bottles of 60: NDC 0456-1510-60

Bottles of 180: NDC 0456-1510-01

### Titration Pack:

4-Week Titration Pack: NDC 0456-1500-55

Blister package containing 55 tablets: 5×12.5-mg tablets, 8×25-mg tablets, and 42×50 mg tablets.

### Storage

The dosage forms described herein should be stored at 25° C. (77° F.), with excursions permitted between 15° C. and 30° C. (between 59° F. and 86° F.).

Milnacipran and its salts can be administered adjunctively with other active compounds such as, for example, analgesics, anti-inflammatory drugs, antipyretics, antidepressants, antiepileptics, antihistamines, antimigraine drugs, antimuscarinics, anxiolytics, sedatives, hypnotics, antipsychotics, bronchodilators, anti asthma drugs, cardiovascular drugs, corticosteroids, dopaminergics, electrolytes, gastro-intestinal drugs, muscle relaxants, nutritional agents, vitamins, parasympathomimetics, stimulants, anorectics and anti-narcoleptics.

Specific examples of compounds that can be adjunctively administered with milnacipran include, but are not limited to, aceclofenac, acetaminophen, adomexetine, almotriptan, alprazolam, amantadine, amcinonide, aminocyclopropane, amitriptyline, amlodipine, amoxapine, amphetamine, aripiprazole, aspirin, atomoxetine, azasetron, azatadine, beclomethasone, benactyzine, benoxaprofen, bermoprofen, betamethasone, bicifadine, bromocriptine, budesonide, buprenorphine, bupropion, buspirone, butorphanol, butriptyline, caffeine, carbamazepine, carbidopa, carisoprodol, celecoxib, chlordiazepoxide, chlorpromazine, choline salicylate, citalopram, clomipramine, clonazepam, clonidine, clonitazene, clorazepate, clotiazepam, cloxazolam, clozapine, codeine, corticosterone, cortisone, cyclobenzaprine, cyproheptadine, demexiptiline, desipramine, desomorphine, dexamethasone, dexanabinol, dextroamphetamine sulfate, dextromoramide, dextropropoxyphene, dezocine, diazepam, dibenzepin, diclofenac sodium, diflunisal, dihydrocodeine, dihydroergotamine, dihydromorphine, dimetacrine, divalproxex, dizatriptan, dolasetron, donepezil, dothiepin, doxepin, duloxetine, ergotamine, escitalopram, estazolam, ethosuximide, etodolac, femoxetine, fenamates, fenoprofen, fentanyl, fludiazepam, fluoxetine, fluphenazine, flurazepam, flurbiprofen, flutazolam, fluvoxamine, frovatriptan, gabapentin, galantamine, gepirone, ginko biloba, granisetron, haloperidol, huperzine A, hydrocodone, hydrocortisone, hydromorphone, hydroxyzine, ibuprofen, imipramine, indiplon, indomethacin, indoprofen, iprindole, ipsapirone, ketaserin, ketoprofen, ketorolac, lesopitron, levodopa, lipase, lofepramine, lorazepam, loxapine, maprotiline, mazindol, mefenamic acid, melatonin, melitracen, memantine, meperidine, meprobamate, mesalamine, metapramine, metaxalone, methadone, methadone, methamphetamine, methocarbamol, methyldopa, methylphenidate, methylsalicylate, methysergid(e), metoclopramide, mianserin, mifepristone, milnacipran, minaprine, mirtazapine, moclobemide, modafinil (an anti-narcoleptic), molindone, morphine, morphine hydrochloride, nabumetone, nadolol, naproxen, naratriptan, nefazodone, neurontin, nomifensine, nortriptyline, olanzapine, olsalazine, ondansetron, opipramol, orphenadrine, oxaflozane, oxaprazin, oxazepam, oxitriptan, oxycodone, oxymorphone, pancrelipase, parecoxib, paroxetine, pemoline, pentazocine, pepsin, perphenazine, phenacetin, phendimetrazine, phenmetrazine, phenylbutazone, phenyloin, phosphatidylserine, pimozide, pirlindole, piroxicam, pizotifen, pizotyline, pramipexole, prednisolone, prednisone, pregabalin, propanolol, propizepine, propoxyphene, protriptyline, quazepam, quinupramine, reboxitine, reserpine, risperidone, ritanserin, rivastigmine, rizatriptan, rofecoxib, ropinirole, rotigotine, salsalate, sertraline, sibutramine, sildenafil, sulfasalazine, sulindac, sumatriptan, tacrine, temazepam, tetrabenozine, thiazides, thioridazine, thiothixene, tiapride, tiasipirone, tizanidine, tofenacin, tolmetin, toloxatone, topiramate, tramadol, trazodone, triazolam, trifluoperazine, trimethobenzamide, trimipramine, tropisetron, valdecoxib, valproic acid, venlafaxine, viloxazine, vitamin E, zimeldine, ziprasidone, zolmitriptan, zolpidem, zopiclone and isomers, salts, and combinations thereof.

In exemplary embodiments, milnacipran, or a pharmaceutically acceptable salt thereof, is administered in combination with gabapentin, pregabalin, pramipexole, 1-DOPA, amphetamine, tizanidine, clonidine, tramadol, morphine, a tricyclic antidepressant, codeine, carbamazepine, sibutramine, valium, carbamazepine or trazadone.

By adjunctive administration is meant simultaneous administration of the compounds, in the same dosage form, simultaneous administration in separate dosage forms, and separate administration of the compounds.

Milnacipran hydrochloride can be administered for the treatment of, for example, fibromyalgia syndrome, chronic fatigue syndrome, pain (e.g., chronic pain, neuropathic pain such as post-herpetic neuralgia, diabetic peripheral neuropathy), attention deficit/hyperactivity disorder, visceral pain syndromes (such as irritable bowel syndrome, noncardiac chest pain, functional dyspepsia, interstitial cystitis, essential vulvodynia, urethral syndrome, orchialgia, affective disorders including depressive disorders (major depressive disorder, dysthymia, atypical depression) and anxiety disorders (generalized anxiety disorder, phobias, obsessive compulsive disorder, panic disorder, post-traumatic stress disorder), premenstrual dysphoric disorder, temperomandibular disorder, atypical face pain, chronic lower back pain, migraine headache, and tension headache.

In exemplary embodiments, milnacipran hydrochloride is administered to treat fibromyalgia syndrome, chronic fatigue syndrome, chronic pain, neuropathic pain (e.g., post-herpetic neuralgia, diabetic peripheral neuropathy) osteoarthritis or chronic back pain.

In some embodiments, the present invention relates to methods of managing fibromyalgia in a patient in need thereof comprising providing about 10 mg to about 200 mg of milnacipran or a pharmaceutically acceptable salt thereof and informing the patient or a health care worker that a recommended dose of milnacipran or a pharmaceutically acceptable salt thereof is about 100 mg/day.

In exemplary embodiments, the present invention provides methods of managing fibromyalgia comprising providing about 10 mg to about 50 mg of milnacipran or a pharmaceutically acceptable salt thereof (e.g., milnacipran hydrochloride) and informing the patient or a health care worker that about 50 mg/day of milnacipran or a pharmaceutically acceptable salt thereof (e.g., milnacipran hydrochloride) should be administered in a patient with a creatinine clearance of about 5 to about 29 ml/min. In further embodiments, the methods comprise informing the patient or a health care worker that about 50 mg of milnacipran or a pharmaceutically acceptable salt thereof (e.g., milnacipran hydrochloride) should be administered in two divided doses per day. The patient or a health care worked may be further informed that about 100 mg/day of milnacipran or a pharmaceutically acceptable salt thereof (e.g., milnacipran hydrochloride) may be administered in a patient with a creatinine clearance of about 5 to about 29 ml/min.

In some embodiments, the methods comprise informing that administration of milnacipran or a pharmaceutically acceptable salt thereof (e.g., milnacipran hydrochloride) with food will lead to an increase in tolerability of the milnacipran or pharmaceutically acceptable salt thereof.

In exemplary embodiments, the methods may comprise informing the patient or a health care about contraindications. For example, the methods may include informing that milnacipran or a pharmaceutically acceptable salt thereof (e.g., milnacipran hydrochloride) is contraindicated in a patient taking a monoamine oxidase inhibitor. Further information that at least 14 days should have elapsed between a discontinuation of a monoamine oxidase inhibitor and an administration of milnacipran or a pharmaceutically acceptable salt thereof may also be provided. In other embodiments, information on adverse reactions resulting from administration of milnacipran or pharmaceutically acceptable salt thereof (e.g., milnacipran hydrochloride) in a patient taking a monoamine oxidase inhibitor may be provided. Examples of such adverse reaction include, but are not limited to, hyperthermia, rigidity, myoclonus, autonomic instability and a mental status change.

In other examples, the methods may include informing that milnacipran or pharmaceutically acceptable salt thereof (e.g., milnacipran hydrochloride) is contraindicated in a patient with uncontrolled narrow-angle glaucoma.

In some embodiments, information on co-administration of milnacipran or a pharmaceutically acceptable salt thereof (e.g., milnacipran hydrochloride) and other agents may be provided. For example, the patient or health care worker may be informed that co-administration of the milnacipran or pharmaceutically acceptable salt thereof (e.g., milnacipran hydrochloride) with lithium may result in serotonin syndrome. In other examples, information that co-administration of the milnacipran or pharmaceutically acceptable salt thereof (e.g., milnacipran hydrochloride) with a serotonin re-uptake inhibitor may result in a condition, such as hypertension and/or coronary artery vasoconstriction may be provided. In other embodiments, the patient or a health care worker may be informed that co-administration of the milnacipran or pharmaceutically acceptable salt thereof (e.g., milnacipran hydrochloride) with epinephrine or norepinephrine may result in a condition, such as paroxysmal hypertension and/or arrhythmia. In still other embodiments, the patient or a health care worker may be informed that co-administration of the milnacipran or pharmaceutically acceptable salt thereof (e.g., milnacipran hydrochloride) with digoxin may result in potentiation of adverse hemodynamic effects.

### Patient Counseling Information

Prescribers or other health professionals should inform patients, their families, and their caregivers about the benefits and risks associated with treatment with milnacipran or pharmaceutically acceptable salt thereof (e.g., milnacipran hydrochloride) and should counsel them in its appropriate use. A patient Medication Guide is available for milnacipran hydrochloride. The prescriber or health professional should instruct patients, their families, and their caregivers to read the Medication Guide and should assist them in understanding its contents. Patients should be given the opportunity to discuss the contents of the Medication Guide and to obtain answers to any questions they may have. The complete text of the Medication Guide is reprinted below.

Patients should be advised of the following issues and asked to alert their prescriber if these occur while taking milnacipran hydrochloride

Patients and their families and caregivers should be advised that milnacipran hydrochloride is a selective norepinephrine and serotonin reuptake inhibitor and therefore belongs to the same class of drugs as antidepressants. Patients, their families and their caregivers should be advised that patients with depression may be at increased risk for clinical worsening and/or suicidal ideation if they stop taking anti-depressant medication, change the dose, or start a new medication.

Patients, their families and their caregivers should be encouraged to be alert to the emergence of anxiety, agitation, panic attacks, insomnia, irritability, hostility, aggressiveness, impulsivity, akathisia (psychomotor restlessness), hypomania or other unusual changes in behavior, worsening of depression, and suicidal ideation, especially early during treatment with milnacipran hydrochloride or other drugs that inhibit the reuptake of norepinephrine and/or serotonin, and when the dose is adjusted up or down. Families and caregivers of patients should be advised to observe for the emergence of such symptoms on a day-to-day basis, since changes may be abrupt. Such symptoms should be reported to the patient's prescriber or health professional, especially if they are severe, abrupt in onset, or were not part of the patient's presenting symptoms.

In certain embodiments, patients should be cautioned about the risk of serotonin syndrome with concomitant use of milnacipran hydrochloride and triptans, tramadol, or other serotonergic agents.

In certain embodiments, patients should be advised that their blood pressure and pulse should be monitored at regular intervals when receiving treatment with milnacipran hydrochloride

In certain embodiments, patients should be cautioned about the concomitant use of milnacipran hydrochloride and NSAIDs, aspirin, or other drugs that affect coagulation, since the combined use of agents that interfere with serotonin reuptake and these agents has been associated with an increased risk of abnormal bleeding.

Milnacipran hydrochloride might diminish mental and physical capacities necessary to perform certain tasks such as operating machinery, including motor vehicles. In certain embodiments, patients should be cautioned about operating machinery or driving motor vehicles until they are reasonably certain that milnacipran hydrochloride treatment does not affect their ability to engage in such activities.

In certain embodiments, patients should be advised to avoid consumption of alcohol while taking milnacipran hydrochloride

In certain embodiments, patients should be advised that withdrawal symptoms can occur when discontinuing treatment with milnacipran hydrochloride particularly when discontinuation is abrupt.

In certain embodiments, patients should be advised to notify their physician if they become pregnant or intend to become pregnant during milnacipran hydrochloride therapy.

In certain embodiments, patients should be advised to notify their physician if they are breast-feeding.

Milnacipran hydrochloride is not used to treat depression, but it acts like medicines that are used to treat depression (antidepressants) and other psychiatric disorders.

In certain embodiments, a patient is advised to read the Medication Guide that comes with the patient's antidepressant medicine. This Medication Guide is only about the risk of suicidal thoughts or actions with antidepressant medicines.

In further embodiments, the patient is advised to talk to the healthcare provider, and/or to ask the healthcare provider about any of the following:

all risks and benefits of treatment with antidepressant medicines;

all treatment choices for depression or other serious mental illness.

What is the most important information I should know about antidepressant medicines, depression and other serious mental illnesses, and suicidal thoughts or actions?

Antidepressant medicines may increase suicidal thoughts or actions in some children, teenagers, and young adults within the first few months of treatment.

Depression and other serious mental illnesses are the most important causes of suicidal thoughts and actions. Some people may have a particularly high risk of having suicidal thoughts or actions. These include people who have (or have a family history of) bipolar illness (also called manic-depressive illness) or suicidal thoughts or actions.

How can I watch for and try to prevent suicidal thoughts and actions in myself or a family member?

Pay close attention to any changes, especially sudden changes, in mood, behaviors, thoughts, or feelings. This is very important when an antidepressant medicine is started or when the dose is changed.

What else do I need to know about antidepressant medicines?

Never stop an antidepressant medicine without first talking to a healthcare provider. Stopping an antidepressant medicine suddenly can cause other symptoms.

Antidepressants are medicines used to treat depression and other illnesses. It is important to discuss all the risks of treating depression and also the risks of not treating it. Patients and their families or other caregivers should discuss all treatment choices with the healthcare provider, not just the use of antidepressants.

Antidepressant medicines have other side effects. Talk to the healthcare provider about the side effects of the medicine prescribed for you or your family member.

Antidepressant medicines can interact with other medicines. Know all of the medicines that you or your family member takes. Keep a list of all medicines to show the healthcare provider. Do not start new medicines without first checking with your healthcare provider.

Not all antidepressant medicines prescribed for children are FDA approved for use in children. Talk to your child's healthcare provider for more information. Call your doctor for medical advice about side effects.

In additional embodiments, a patient is advised to call the healthcare provider right away to report new or sudden changes in mood, behavior, thoughts, or feelings.

In additional embodiments, a patient is advised to keep all follow-up visits with the healthcare provider as scheduled. In additional embodiments, a patient is advised to call the healthcare provider between visits as needed, especially if the patient has concerns about symptoms.

In additional embodiments, a patient or a family member thereof is advised to call the health care provider right away if the patient has any of the following symptoms, especially if they are new, worse, or worry the patient or family member:

thoughts about suicide or dying
attempts to commit suicide
new or worse depression
new or worse anxiety
feeling very agitated or restless
panic attacks
new or worse irritability
acting aggressive, being angry, or violent
acting on dangerous impulses
an extreme increase in activity and talking (mania)
other unusual changes in behavior or mood
trouble sleeping (insomnia)

### DEFINITIONS

The term “treating” means to relieve, alleviate, delay, reduce, reverse, improve, manage or prevent at least one symptom of a condition in a subject. The term “treating” may also mean to arrest, delay the onset (i.e., the period prior to clinical manifestation of a disease) and/or reduce the risk of developing or worsening a condition.

A subject or patient in whom administration of the therapeutic compound is an effective therapeutic regimen for a disease or disorder is preferably a human, but can be any animal, including a laboratory animal in the context of a clinical trial or screening or activity experiment. Thus, as can be readily appreciated by one of ordinary skill in the art, the methods, compounds and compositions of the present invention are particularly suited to administration to any animal, particularly a mammal, and including, but by no means limited to, humans, domestic animals, such as feline or canine subjects, farm animals, such as but not limited to bovine, equine, caprine, ovine, and porcine subjects, wild animals (whether in the wild or in a zoological garden), research animals, such as mice, rats, rabbits, goats, sheep, pigs, dogs, cats, etc., avian species, such as chickens, turkeys, songbirds, etc., i.e., for veterinary medical use.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per practice in the art. Alternatively, “about” with respect to the compositions can mean plus or minus a range of up to 20%, preferably up to 10%, more preferably up to 5%.

“Substantially pure” enantiomers contain no more than about 5% w/w of the corresponding opposite enantiomer, such as no more than about 2%, no more than about 1% or no more than about 0.5% w/w.

The pharmacokinetic parameters described herein include area under the plasma concentration-time curve (AUC₀₋ₜ and AUC_{0-∞}), maximum plasma concentration (Cₘₐₓ), time of maximum plasma concentration (Tₘₐₓ) and terminal elimination half-life (T_{1/2}). The time of maximum concentration, Tₘₐₓ, is determined as the time corresponding to Cₘₐₓ. Area under the plasma concentration-time curve up to the time corresponding to the last measurable concentration (AUC₀₋ₜ) is calculated by numerical integration using the linear trapezoidal rule as follows:

$${AUC}_{0 - t} = {\sum }_{i = 2}^{n} 0.5 \cdot ( {C}_{i} + {C}_{i - 1} ) \cdot ( {t}_{i} - {t}_{i - 1} ) \qquad Eq . 1$$

where Cᵢ is the plasma milnacipran concentrations at the corresponding sampling time point tᵢ and n is the number of time points up to and including the last quantifiable concentration.

The terminal half-life (T_{1/2}) is calculated using the following equation:

$${T}_{1 / 2} = \frac{0.693}{{λ}_{z}} \qquad Eq . 2$$

where λ_{z} is the terminal elimination rate constant.

The area under the plasma concentration-time curve from time zero to infinity is calculated according to the following equation:

$${AUC}_{0 - ∞} = {AUC}_{0 - t} + \frac{{C}_{last}}{{λ}_{z}} \qquad Eq . 3$$

where Cₗₐₛₜ is the last measurable concentration.

### EXAMPLES

The following examples are merely illustrative of the present invention and should not be construed as limiting the scope of the invention in any way as many variations and equivalents that are encompassed by the present invention will become apparent to those skilled in the art upon reading the present disclosure.

### Example 1

### Clinical Studies

### Management of Fibromyalgia

The efficacy of milnacipran hydrochloride for the management of fibromyalgia was established in two double-blind, placebo-controlled, multicenter studies in adult patients (18-74 years of age). Enrolled patients met the American College of Rheumatology (ACR) criteria for fibromyalgia (a history of widespread pain for 3 months and pain present at 11 or more of the 18 specific tender point sites). Approximately 35% of patients had a history of depression. Study 1 was six months in duration and Study 2 was three months in duration.

A larger proportion of patients treated with milnacipran hydrochloride than with placebo experienced a simultaneous reduction in pain from baseline of at least 30% (VAS) and also rated themselves as much improved or very much improved based on the patient global assessment (PGIC). In addition, a larger proportion of patients treated with milnacipran hydrochloride met the criteria for treatment response, as measured by the composite endpoint that concurrently evaluated improvement in pain (VAS), physical function (SF-36 PCS), and patient global assessment (PGIC), in fibromyalgia as compared to placebo.

### Study 1

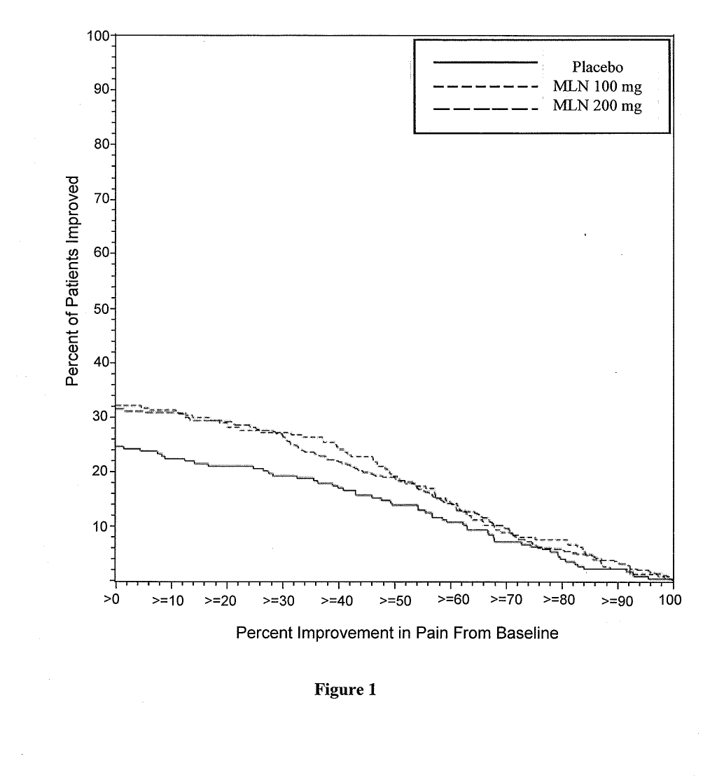

This 6-month study compared total daily doses of milnacipran hydrochloride 100 mg and 200 mg to placebo. Patients were enrolled with a minimum mean baseline pain score of ≧50 mm on a 100 mm visual analog scale (VAS) ranging from 0 (“no pain”) to 100 (“worst possible pain”). The mean baseline pain score in this trial was 69. The efficacy results for Study 1 are summarized in FIG. 1, which shows the proportion of patients achieving various degrees of improvement in pain from baseline to the 3-month time point and who concurrently rated themselves globally improved (PGIC score of 1 or 2). Patients who did not complete the 3-month assessment were assigned 0% improvement. More patients in the milnacipran hydrochloride treatment arms experienced at least a 30% reduction in pain from baseline (VAS) and considered themselves globally improved (PGIC) than did patients in the placebo arm. Treatment with milnacipran hydrochloride 200 mg/day did not confer greater benefit than treatment with milnacipran hydrochloride 100 mg/day.

### Study 2

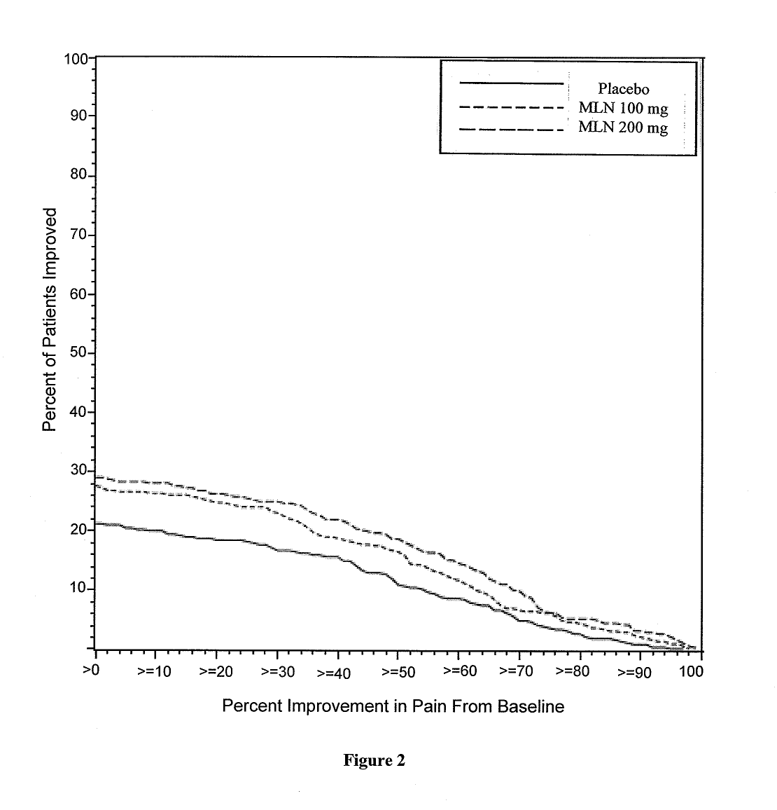

This 3-month study compared total daily doses of milnacipran hydrochloride 100 mg and 200 mg to placebo. Patients were enrolled with a minimum mean baseline pain score of ≧40 mm on a 100-mm VAS ranging from 0 (“no pain”) to 100 (“worst possible pain”). The mean baseline pain score in this trial was 65. The efficacy results for Study 2 are summarized in FIG. 2, which shows the proportion of patients achieving various degrees of improvement in pain from baseline to the 3-month time point and who concurrently rated themselves globally improved (PGIC score of 1 or 2). Patients who did not complete the 3-month assessment were assigned 0% improvement. More patients in the milnacipran hydrochloride treatment arms experienced at least a 30% reduction in pain from baseline (VAS) and considered themselves globally improved (PGIC) than did patients in the placebo arm. Treatment with milnacipran hydrochloride 200 mg/day did not confer greater benefit than treatment with milnacipran hydrochloride 100 mg/day.

In both studies, some patients who rated themselves as globally “much” or “very much” improved experienced a decrease in pain as early as week 1 of treatment with a stable dose of milnacipran hydrochloride that persisted throughout these studies.

### Example 2

FIG. 3 represents a titration pack comprising milnacipran hydrochloride tablets of three different strengths: 12.5 mg, 25 mg, and 50 mg for oral administration over a period of two weeks. The patient is provided instructions on dosage and administration of milnacipran hydrochloride and advised to titrate the dosage according to the following schedule: Day 1: 12.5 mg requiring a single administration of 12.5 mg tablet; Days 2-3: 25 mg/day requiring administration of a 12.5 mg milnacipran hydrochloride tablet twice a day, Days 4-7: 50 mg/day requiring administration of a 25 mg milnacipran hydrochloride tablet twice a day, and after Day 7: 100 mg/day requiring administration of a 50 mg milnacipran hydrochloride tablet twice a day. Depending on the response after Day 7, if necessary, the patient is optionally advised to titrate the dosage to 200 mg/day. The patient is informed that the recommended dosage is about 100 mg to 200 mg daily.

### Example 3

FIG. 4 represents a titration pack comprising milnacipran hydrochloride tablets of three different strengths: 12.5 mg, 25 mg and 50 mg for oral administration over a period of four weeks. The patient is provided instructions on dosage and administration of milnacipran hydrochloride and advised to titrate the dosage according to the following schedule: Day 1: 12.5 mg requiring a single administration of 12.5 mg tablet; Days 2-3: 25 mg/day requiring administration of a 12.5 mg milnacipran hydrochloride tablet twice a day, Days 4-7: 50 mg/day requiring administration of a 25 mg milnacipran hydrochloride tablet twice a day, Days 8-28: 100 mg/day requiring administration of a 50 mg milnacipran hydrochloride tablet twice a day. Depending on the response after Day 28, if necessary, the patient is optionally advised to titrate the dosage to 200 mg/day. The patient is informed that the recommended dosage is about 100 mg to 200 mg daily.

### Example 4

FIG. 5 represents a titration pack comprising milnacipran hydrochloride tablets of three different strengths: 12.5 mg, 25 mg and 50 mg for oral administration over a period of three weeks. The patient is provided instructions on dosage and administration of milnacipran hydrochloride and advised to titrate the dosage according to the following schedule: Day 1: 12.5 mg requiring a single administration of a 12.5 mg tablet; Days 2-3: 25 mg/day requiring administration of 12.5 mg milnacipran hydrochloride tablet twice a day, Days 4-7: 50 mg/day requiring administration of a 25 mg milnacipran hydrochloride tablet twice a day, Days 8-14: 100 mg/day requiring administration of a 50 mg milnacipran hydrochloride tablet twice a day, Days 15-21: 200 mg/day requiring administration of two 50 mg milnacipran hydrochloride tablets twice a day. The patient is informed that the recommended dosage is about 100 mg to 200 mg daily.

### Example 5

FIG. 6 represents a titration pack comprising milnacipran hydrochloride tablets of four different strengths: 12.5 mg, 25 mg, 50 mg, and 100 mg for oral administration over a period of three weeks. The patient is provided instructions on dosage and administration of milnacipran hydrochloride, and advised to titrate the dosage according to the following schedule: Day 1: 12.5 mg requiring a single administration of a 12.5 mg tablet; Days 2-3: 25 mg/day requiring administration of a 12.5 mg milnacipran hydrochloride tablet twice a day, Days 4-7: 50 mg/day requiring administration of a 25 mg milnacipran hydrochloride tablet twice a day, Days 8-14: 100 mg/day requiring administration of two 50 mg milnacipran hydrochloride tablets twice a day; Days 15-21: 200 mg/day requiring administration of a 100 mg milnacipran hydrochloride tablet twice a day The patient is informed that the recommended dosage is about 100 mg to 200 mg daily.

The entire disclosures of all applications, patents and publications, cited above and below, are hereby incorporated by reference.

While the invention has been depicted and described by reference to exemplary embodiments of the invention, such a reference does not imply a limitation on the invention, and no such limitation is to be inferred. The invention is capable of considerable modification, alteration, and equivalents in form and function, as will occur to those ordinarily skilled in the pertinent arts having the benefit of this disclosure. The depicted and described embodiments of the invention are exemplary only, and are not exhaustive of the scope of the invention. Consequently, the invention is intended to be limited only by the spirit and scope of the appended claims, giving full cognizance to equivalence in all respects.

What is claimed:

1. A method of managing fibromyalgia in a patient in need thereof comprising administering a dose of about 10 mg to about 50 mg of milnacipran or a pharmaceutically acceptable salt thereof per day wherein the patient has a creatinine clearance of about 5 to about 29 ml/min.

2. The method according to claim 1, wherein the method comprises administering milnacipran or a pharmaceutically acceptable salt thereof in a dose of about 12.5 mg on Day 1, about 25 mg on Days 2-3, about 50 mg on Day 4 and maintaining the dose at about 50 mg per day after Day 4.

3. The method according to claim 1, wherein the dose is about 50 mg per day.

4. The method according to claim 3, wherein the dose is administered in two divided doses of about 25 mg.

5. The method according to claim 1, wherein the method comprises administering milnacipran hydrochloride.

6. A method of managing fibromyalgia in a patient in need thereof comprising providing about 10 mg to about 50 mg of milnacipran or a pharmaceutically acceptable salt thereof and informing the patient or a health care worker that about 50 mg/day of milnacipran or a pharmaceutically acceptable salt thereof should be administered in a patient with a creatinine clearance of about 5 to about 29 ml/min.

7. The method according to claim 6, further comprising informing the patient or a health care worker that about 50 mg of milnacipran or a pharmaceutically acceptable salt thereof should be administered in two divided doses per day.

8. The method according to claim 6, further comprising informing the patient or a health care worker that up to about 100 mg/day of milnacipran or a pharmaceutically acceptable salt thereof may be administered in a patient with a creatinine clearance of about 5 to about 29 ml/min.

9. The method according to claim 6, further comprising informing the patient or a health care worker that an administration of milnacipran or a pharmaceutically acceptable salt thereof with food will lead to an increase in tolerability of the milnacipran or pharmaceutically acceptable salt thereof.

10. The method according to claim 6, further comprising informing the patient or a health care worker that milnacipran or a pharmaceutically acceptable salt thereof is contraindicated in a patient taking a monoamine oxidase inhibitor.

11. The method according to claim 10, further comprising informing the patient or a health care worker that at least 14 days should have elapsed between a discontinuation of a monoamine oxidase inhibitor and an administration of milnacipran or a pharmaceutically acceptable salt thereof.

12. The method according to claim 6, further comprising informing the patient or a health care worker that an administration of the milnacipran or pharmaceutically acceptable salt thereof in a patient taking a monoamine oxidase inhibitor may result in an adverse reaction selected from the group consisting of hyperthermia, rigidity, myoclonus, autonomic instability and a mental status change.

13. The method according to claim 6, further comprising informing the patient or a health care worker that co-administration of the milnacipran or pharmaceutically acceptable salt thereof with lithium may result in serotonin syndrome.

14. The method according to claim 6, further comprising informing the patient or a health care worker that co-administration of the milnacipran or pharmaceutically acceptable salt thereof with a serotonin re-uptake inhibitor may result in a condition selected from the group consisting of hypertension and coronary artery vasoconstriction.

15. The method according to claim 6, further comprising informing the patient or a health care worker that co-administration of the milnacipran or pharmaceutically acceptable salt thereof with epinephrine or norepinephrine may result in a condition selected from the group consisting of paroxysmal hypertension and arrhythmia.

16. The method according to claim 6, further comprising informing the patient or a health care worker that co-administration of the milnacipran or pharmaceutically acceptable salt thereof with digoxin may result in potentiation of adverse hemodynamic effects.

17. The method according to claim 6, further comprising informing the patient or a health care worker that milnacipran or pharmaceutically acceptable salt thereof is contraindicated in a patient with uncontrolled narrow-angle glaucoma.
